(19) United States
(12) Patent Application Publication
(10) Pub. No.: US 2014/0081369 A1
(43) Pub. Date: Mar. 20, 2014

(54) HEADACHE-TREATMENT DEVICE WITH GEL DISPENSING KIT AND METHOD

(71) Applicants: Victor Manuel Valencia Sosa, Mexico City (MX); Viridiana Rodríguez Bermudez, Mexico City (MX); Juan Manuel González Suárez, Mexico City (MX); Omar Mendizabal Riveros, Mexico City (MX); Rocío Guzmán, Mexico City (MX); Alejandro Covalin, Culver City, CA (US)

(72) Inventors: Victor Manuel Valencia Sosa, Mexico City (MX); Viridiana Rodríguez Bermudez, Mexico City (MX); Juan Manuel González Suárez, Mexico City (MX); Omar Mendizabal Riveros, Mexico City (MX); Rocío Guzmán, Mexico City (MX); Alejandro Covalin, Culver City, CA (US)

(73) Assignee: Alejandro Covalin, Culver City (CA)

(21) Appl. No.: 14/117,026

(22) PCT Filed: May 11, 2012
(86) PCT No.: PCT/US12/37666

§ 371 Date: Nov. 11, 2013

Related U.S. Application Data
(60) Provisional application No. 61/554,971, filed on Nov. 2, 2011.
(60) Provisional application No. 61/542,792, filed on Oct. 3, 2011.
(60) Provisional application No. 61/485,125, filed on May 11, 2011.

(51) Int. Cl. A61N 1/04; B65D 83/00

(52) U.S. Cl. CPC A61N 1/0456; B65D 83/0055
USPC 607/139; 222/92

(57) ABSTRACT

Electrical-stimulation device with gel-dispensing kit, and a method of making and using the parts of the kit. A convenient and easy-to-use system to provide an electrically conductive path from a transcutaneous electrical nerve stimulation (TENS) device to the skin surface of a patient to supply transcutaneous stimulation, even through hair. The invention provides improved prevention and treatment for headache, depression, alertness, attention deficit hyperactivity disorder (ADHD), epilepsy, anxiety, post-traumatic stress disorder (PTSD), and behavioral and/or other disorders. Some embodiments provide a headache-treatment system that includes an electrode base shaped to conform to a back of a human head; a TENS having projecting spring electrodes each connected to the electrode base; means for holding an electrically conductive gel in a plurality of sealed pockets; and means for unsealing the means for holding the gel and applying the gel substantially simultaneously to the projecting spring electrodes.

### CROSS-REFERENCE TO RELATED APPLICATIONS

This is a U.S. national-stage patent application under 35 USC §371 from, and claims priority benefit under 35 USC §365(a)-(c) of, PCT Application No. PCT/US2012/037666 filed May 11, 2012 and titled “HEADACHE-TREATMENT DEVICE WITH GEL DISPENSING KIT AND METHOD,” which claims priority benefit of U.S. Provisional Patent Application No. 61/485,125 filed May 11, 2011 by Victor Manuel Valencia Sosa et al., titled “HEADACHE-TREATMENT DEVICE WITH GEL DISPENSING KIT AND METHOD”; and claims priority benefit of U.S. Provisional Patent Application No. 61/542,792 filed Oct. 3, 2011 by Alejandro Covalin, titled “METHOD FOR TREATING HEADACHE, NEUROPSYCHIATRIC AND/OR NEUROLOGICAL CONDITIONS OR DISORDERS AND TREATMENT DEVICE WITH GEL DISPENSING KIT”; and claims priority benefit of U.S. Provisional Patent Application No. 61/554,971 filed Nov. 2, 2011 by Alejandro Covalin, titled “METHOD OF TREATMENT FOR AN ALERTNESS CONDITION OR DISORDER, AND ADHD, AND TREATMENT DEVICE WITH GEL DISPENSING KIT”; all of which non-provisional and provisional patent applications are incorporated herein in their entirety by reference.

This invention is related to: U.S. Provisional Patent Application No. 60/911,518 by Alejandro Covalin, filed Apr. 13, 2007, titled “APPARATUS AND METHOD FOR THE TREATMENT OF HEADACHE”; PCT Patent Application PCT/US2008/060288 by Alejandro Covalin, filed Apr. 14, 2008, titled “APPARATUS AND METHOD FOR THE TREATMENT OF HEADACHE”; U.S. patent application Ser. No. 12/578,369 by Alejandro Covalin, filed Oct. 13, 2009, titled “APPARATUS AND METHOD FOR THE TREATMENT OF HEADACHE”; U.S. Provisional Patent Application No. 61/020,111 by Alejandro Covalin et al., filed Jan. 9, 2008, titled “SYSTEM AND METHOD FOR THE MODULATION OF FEEDING AND MILK PRODUCTION”; and U.S. patent application Ser. No. 12/350,497 by Alejandro Covalin et al., filed Jan. 8, 2009, titled “SYSTEM AND METHOD FOR THE MODULATION OF FEEDING AND MILK PRODUCTION”; which are all incorporated herein in their entirety by reference.

### FIELD OF THE INVENTION

This invention relates to methods and apparatus for the treatment of headache, neuropsychiatric, neurological, and alertness conditions or disorders, and/or attention-deficit-hyperactivity disorder (ADHD). More specifically, this invention relates to a method and a portable, (e.g., battery-operated) transcutaneous electrical nerve stimulation (TENS) device for the treatment of headache, neuropsychiatric, neurological, and alertness conditions or disorders, and/or attention deficit hyperactivity disorder (ADHD), as well as an applicator and method for applying a gel electrolyte to the electrodes of the TENS headache-treatment device to the occipital, and optionally to the supraorbital and/or temporal regions of a patient's head as well.

### BACKGROUND OF THE INVENTION

Headache is so common that it is often not regarded as a serious health issue. However, more than 45 million Americans suffer from headaches severe enough to seek medical help, and people around the world would benefit from an improved treatment regimen for headache. A study published in the April 1999 issue of the Archives of Internal Medicine revealed that one single headache type, namely migraine, costs American employers $13 billion per year due to missed work and reduced productivity. In addition, according to the National Headache Foundation (NHF), approximately 157 million workdays are lost annually due to the pain and the associated symptoms of migraine. On top of the above mentioned economic impact, there are intangible costs, such the poor quality of life of these headache sufferers due to the time missed from their daily activities.

The International Headache Society (IHS) classifies headache disorders into two main categories: primary and secondary. The main difference between these two categories is that the secondary type headaches are attributed to a particular cause. Although the overall mechanisms and specific pathways responsible for primary and secondary headaches are still being elucidated, it is well known that many patients that experience these types of headaches often feel pain in both the front and the back of the head. While the front of the head is innervated, among others, by the ophthalmic branch of the trigeminal nerve, the back of the head is mainly innervated by spinal branches arising from C1 through C4, which are the first four cervical spinal nerves and which, among others, form the occipital and suboccipital nerves. However, recent studies suggest that there is a functional connection between the branches of the nervous system that innervate the front and the back of the head.

The most commonly used treatment to mitigate headache pain is to date the pharmacological approach, which, depending on the particular drug, according to the American Council for Headache Education and to the National Guideline Clearinghouse (NGC) has various potential side effects. Some of these side effects include: fatigue, depression, nausea, insomnia, weight gain, constipation, dizziness, low blood pressure, gastrointestinal irritation, impaired platelet function, renal complications, analgesic rebound headache, and hepatic complications.

Studies exploring alternative treatments for headaches have shown that electrically stimulating spinal nerve branches arising from between C1 through C4 provide an effective technique to mitigate several types of headaches. Electrostimulation of other sites in the head has also been successfully used to treat headaches. A study by Solomon et al. (“Treatment of Headache by Transcutaneous Electrical Stimulation,” Headache, Volume 25, pages 12-15, 1985) used high frequency (12 kHz to 20 kHz) transcutaneous electrical stimulation (TENS) in which one electrode was placed over the area of maximum pain and a second electrode on the opposite side of the head. In the same study, in cases where the pain was generalized, one electrode was placed in the occiput (back of the head) and another on the right hand. Solomon et al. found that fifty-five percent (55%) of patients noted an improvement after electrostimulation as compared to eighteen percent (18%) after application of a placebo.

In another study by Ahmed et al. (“Use of Percutaneous Electrical Nerve Simulation (PENS) in the Short-term Management of Headache,” Headache, Volume 40, pages 311-315, 2000), percutaneous electrical nerve stimulation (PENS) was used to treat tension-type headache (TTH), migraine and posttraumatic headache (PTH). This study revealed that, regardless of the type of headache, pain was significantly mitigated; 58%, 59%, and 52% in TTH, migraine, and PSH respectively. On top of the pain reduction, a reduction in the frequency of headaches was also observed.

As mentioned above, electrostimulation of the spinal branches arising from C1 through C4 in the occipital and suboccipital region has proven to be effective in mitigating pain and reducing the frequency of occurrence of several types of headaches. However, the approach taken involves the chronic implantation of electrodes into the aforementioned anatomical region along with the implantation of a stimulating unit (sometimes referred to as an implantable pulse generator (IPG)), in a second location or in the same region (e.g., U.S. Pat. No. 6,735,475 to Whitehurst et al. on May 11, 2004, titled “Fully Implantable Miniature Neurostimulator for Stimulation as a Therapy for Headache and/or Facial Pain,” which is incorporated herein by reference) to produce the stimulating signal. Several scientific publications assess the effectiveness of this approach (e.g., Matharu et al., “Central neuromodulation in chronic migraine patients with suboccipital stimulators: a PET study,” Brain, Volume 127, pages 220-230, 2004; Popeney et al., “Peripheral Neurostimulation for the Treatment of Chronic, Disabling Transformed Migraine,” Headache, Volume 43, pages 369-375, 2003; Schwedt et al., “Response to occipital nerve block is not useful in predicting efficacy of occipital nerve stimulation,” Cephalalgia, Volume 27, pp. 271-274, 2007(a); Schwedt et al., “Occipital nerve stimulation for chronic headache—long-term safety and efficacy,” Cephalalgia, Volume 27, pp. 153-157, 2007(b); Schwedt, et al., 2007b; Rodrigo-Royo et al., “Peripheral Neurostimulation in the Management of Cervicogenic Headache: Four Case Reports,” Neuromodulation, Volume 8, No. 4, pages 241-248, 2005).

U.S. Pat. No. 5,078,928 to Balster et al. issued Jan. 7, 1992, titled “COATING PROCESS FOR MANUFACTURING ENLARGED SMOOTH TEETH ENDS ON COMB” (hereinafter, “Balster et al.”), describes a process that could be used to manufacture smooth, comb-like electrodes, and is incorporated herein by reference.

Japanese Patent Publication No. 08-173551 by Oshio Kozo, published on Jul. 9, 1996, is titled “ELECTRODE FOR SCALP ELECTROMASSAGE” (hereinafter, “Kozo”). Kozo describes an electrode body for a scalp massage connected to ends of lead wires with a base board formed of a plastic material into a disk shape. The base board is formed into a curved shape for fitting a scalp surface shape. Electrode pins are embedded in an interior surface of the base board. The electrode pin is formed of stainless steel or the like, with its tip formed into a spherical shape, and protruding from the base board. The tips of respective electrode pins form a curved surface parallel to the curved surface of the base board. The electrode pins are classified into two groups; different kinds of poles are connected to each other to make a conductive state, and connected to lead wires.

U.S. Patent Application No. US 2010/0030299 published Feb. 4, 2010 titled “APPARATUS AND METHOD FOR THE TREATMENT OF HEADACHE” is incorporated herein in its entirety by reference. This document describes a battery-operated transcutaneous electrical nerve stimulator (TENS) to treat headache pain in an abortive and/or preventive manner. The TENS unit and its electrodes are built into a unitary device which facilitates a self-administered treatment. An internal gel-dispensing embodiment is described wherein the gel is extruded through hollow electrodes to the conductive ends of the electrodes.

U.S. Pat. No. 5,281,453 issued to Yamada et al. on Jan. 25, 1994, titled “MULTILAYER COMPOSITES AND EASILY OPENABLE CONTAINERS,” which is incorporated by reference, describes a multilayer composite and an easily openable container produced by using the multilayer composite. The multilayer composite includes a surface layer of a polyolefin resin, an intermediate layer of a mixture of a polystyrenic resin with a thermoplastic elastomer or with a thermoplastic elastomer and a polyolefin resin, and a base layer of a polystyrenic resin, and the peeling strength between the intermediate layer and the base layer is larger than the peeling strength between the surface layer and the intermediate layer. The easily openable container produced by using the multilayer composite is capable of being sealed with a lid strongly while maintaining easy openability because the opening of the sealed container is performed utilizing the peeling between the surface layer and the intermediate layer.

U.S. Patent Application 2011/0106220 by DeGiorgio et al. published May 5, 2011 titled “Extracranial Implantable Devices, Systems and Methods for the Treatment of Neurological Disorders” and is incorporated herein by reference. DeGiorgio et al. describe methods, devices and systems used for the treatment of neurological disorders via stimulation of the superficial elements of the trigeminal nerve (“TNS”). More specifically, minimally invasive methods of stimulation of the superficial branches of the trigeminal nerve located extracranially in the face, namely the supraorbital, supratrochlear, infratrochlear, auriculotemporal, zygomaticotemporal, zygomaticoorbital, zygomaticofacial, nasal, infraorbital, and mentalis nerves (also referred to collectively as the superficial trigeminal nerve) are disclosed therein. Systems and devices configured for therapeutic stimulation of the branches of the trigeminal nerves, such as the superficial trigeminal nerve, and their methods of application are described.

U.S. Patent Application 2011/0112603 by DeGiorgio et al. published May 12, 2011 titled “Extracranial Implantable Devices, Systems and Methods for the Treatment of Neuropsychiatric Disorders” and is incorporated herein by reference. There, DeGiorgio et al. describe methods, devices, and systems used for the treatment of mood, anxiety, cognitive, and behavioral disorders (collectively, neuropsychiatric disorders) via stimulation of the superficial elements of the trigeminal nerve (“TNS”).

U.S. Patent Application 2011/0218589 by DeGiorgio et al. published Sep. 8, 2011 titled “Systems, Devices and Methods for the Treatment of Neurological Disorders and Conditions” and is incorporated herein by reference. Here, DeGiorgio et al. describe methods, devices, and systems used for the treatment of and/or promoting recovery from various neurological disorders and conditions, including epilepsy and other seizure disorders and movement and other related disorders; for promoting recovery from acute or chronic brain injury (e.g. stroke, hypoxia/ischemia, head trauma, subarachnoid hemorrhage, and other forms of brain injury, for awakening and/or promoting the recovery of patients in various levels of coma, altered mental status or vegetative state); or for promoting recovery from chronic daily headache and migraine and related disorders via external (cutaneous) stimulation of the sensory branches of the trigeminal nerve in the face and forehead.

U.S. Patent Application 2011/0218590 by DeGiorgio et al. published Sep. 8, 2011 titled “Devices, Systems and Methods for treatment of Neuropsychiatric Disorders” and is incorporated herein by reference. In this patent application, DeGiorgio et al. describe methods, devices and systems used for the treatment of mood, anxiety, post traumatic stress disorder, and cognitive and behavioral disorders (collectively, neuropsychiatric disorders) via stimulation of the superficial elements of the trigeminal nerve (“TNS”).

U.S. Patent Application 2011/0218589 published on Sep. 8, 2011 titled “Systems, Devices and Methods for the Treatment of Neurological Disorders and Conditions” is incorporated herein in its entirety by reference. In this application DeGiorgio et al. describe external (cutaneous) stimulation of the sensory branches of the trigeminal nerve in the face and forehead, and electrode assemblies configured for stimulation of the supraorbital, supratrochlear, infraorbital, auriculotemporal, zygomaticotemporal, zygomaticoorbital, zygomaticofacial, nasal and infratrochlear nerves.

U.S. Patent Application 2011/0218590 published on Sep. 8, 2011 titled “Devices, Systems and Methods for Treatment of Neuropsychiatric Disorders” and is incorporated herein in its entirety by reference. This application described stimulation of the superficial elements of the trigeminal nerve (“TNS”), more specifically, cutaneous methods of stimulation of the superficial branches of the trigeminal nerve located extracranially in the face.

U.S. Pat. No. 5,374,283 to Flick issued Dec. 20, 1994 titled “Electrical therapeutic apparatus” and is incorporated herein in its entirety by reference. Flick described an electrical therapeutic apparatus for the treatment of body pain and edema. The apparatus has an electrical pulse-producing device coupled to a wrap by conductor. The wrap includes nylon coated with silver which forms an electrode. A second electrode is coupled by conductors to the device.

Reed et al. (“Combined occipital and supraorbital neurostimulation for the treatment of chronic migraine headaches: Initial experience,” Cephalalgia, Volume 30(3), pages 260-271, 2010) describe an approach to the treatment of chronic migraine headaches based on neurostimulation of both occipital and supraorbital nerves that was developed and reduced to clinical practice in a series of patients with headaches unresponsive to currently available therapies. Seven patients with chronic migraine and refractory chronic migraine headaches had permanent combined occipital nerve-supraorbital nerve neurostimulation systems implanted.

There is a need for improved headache-treatment systems, particularly headache-treatment systems having improved usability and effectiveness. Many of the patient population suffering from headaches would benefit from, and what is needed are a portable device and a side-effect-free (e.g., non-pharmaceutical) yet effective non-invasive treatment method that could be self administered via the portable device that can be used in both prophylactic and abortive ways of headache treatment. A convenient and easy-to-use system is needed to provide an effective and reliable electrical-conductive path from a TENS device to the skin surface of a patient to supply transcutaneous stimulation.

### BRIEF SUMMARY OF THE INVENTION

The invention herein disclosed describes a non-invasive apparatus and method for the acute treatment of primary and secondary type headaches via transcutaneous electrostimulation of the spinal nerve branches and/or sub branches and/or any of their combinations such as those arising from C1 through C4, including, but not limited to, the right and/or left suboccipital nerve(s), the right and/or left greater occipital nerve(s), the right and/or left least (third) occipital nerve(s), the right and/or left lesser occipital nerve(s), the right and/or left great auricular nerve(s). In some embodiments the non-invasive apparatus and method for the acute treatment of headaches also includes transcutaneous electrostimulation of the supraorbital and/or auriculotemporal superficial trigeminal nerves. In some embodiments, the present invention provides both occipital nerve stimulation and trigeminal nerve stimulation simultaneously, near simultaneously, or in an alternating sequence, providing noninvasive treatment to the trigeminal cervical complex in the brain stem.

In some embodiments, the apparatus includes a device where the stimulating spring electrodes and the stimulator are integrated into a battery-operated transcutaneous electrical nerve stimulating (TENS) device. The aforementioned TENS device can be, but does not have to be, operated by the patient himself or herself. As used herein, the term patient is used to describe any user of the TENS device without implying the user is under the direct care of a physician or therapist. In some embodiments, the apparatus includes a device where the stimulating spring electrodes and the stimulator are integrated into a single hands free battery-operated transcutaneous-electrical nerve-stimulating (TENS) device. In some such embodiments, the patient is free to move and his/her hands are free to do other tasks while the stimulation is being applied. The aforementioned TENS device can be, but does not have to be, operated by the patient himself or herself. In some embodiments, each electrode described herein includes a conductive mesh instead of or in addition to the metal electrode end.

In some embodiments, the present invention provides an apparatus and method for applying gel to absorbent material affixed to the ends of electrically conductive spring electrodes. In some embodiments, the absorbent material is a sponge or sponge-like material that holds the applied gel at the interface between the electrically conductive spring electrodes (e.g., in some embodiments, stainless steel conductors held in a spring configuration within polymer teeth) and the scalp of the patient being treated for headache. In some embodiments, the invention includes a kit having one or more foil-covered blister packs, each blister pack having a one or more pockets, each containing a gel held in a blister-shaped pocket that has been formed in a polymer substrate and sealed in place by a foil or metalized polymer film cover. In some embodiments, the foil side of one such blister pack is pressed simultaneously against the plurality of electrically conductive spring electrodes in order to puncture the foil covering and thus apply the gel substantially simultaneously to the sponge material on the electrode tips. In some other embodiments, the “blister pack” includes an easily peelable layer that is removed to expose the gel, and the opened blister pack is then pressed against the plurality of electrically conductive spring electrodes in order to apply the gel substantially simultaneously to the sponge material on the electrode tips. In yet some other embodiments, blister pack has an easily peelable layer that is removed to expose a thin foil covering the gel, and the blister pack is then pressed against the plurality of electrically conductive spring electrodes in order to puncture the foil covering and thus apply the gel substantially simultaneously to the sponge material on the electrode tips.

### BRIEF DESCRIPTION OF THE DRAWINGS

FIG. 1A is a back-left-side perspective view that illustrates a head piece 110 of a headache-treatment device according to some embodiments of the invention.

FIG. 1B is a front-left-side perspective view of head piece 110 (shown in FIG. 1A) according to some embodiments of the invention.

FIG. 1C is a back perspective view of head piece 110 (shown in FIG. 1A) according to some embodiments of the invention.

FIG. 1D is a left-side perspective view of head piece 110 (shown in FIG. 1A) according to some embodiments of the invention.

FIG. 1E is a top perspective view of head piece 110 (shown in FIG. 1A) according to some embodiments of the invention.

FIG. 2A is a right-side perspective view that illustrates head piece 110 (shown in FIG. 1A) in place on the occiput (back of the head) of patient 99 according to some embodiments.

FIG. 2B is a back-left-side perspective view that illustrates head piece 110 in place on the occiput of patient 99 according to some embodiments of the invention.

FIG. 2C is a front-left-side perspective view that illustrates head piece 110 in place on the occiput of patient 99 according to some embodiments of the invention.

FIG. 2D is a front-left-side perspective view that illustrates head piece 110 with adjustable head strap 120 loosened for adjustment according to some embodiments.

FIG. 3A is a perspective view of headache-treatment device 100 that includes head piece 110, module 320, and electrical cable 130 according to some embodiments.

FIG. 3B is another perspective view of headache-treatment device 100 that includes head piece 110, module 320, and electrical cable 130 according to some embodiments.

FIG. 4A is perspective view of headache-treatment device 100 that includes head piece 110, strap 120, and electrodes 142 according to some embodiments of the invention.

FIG. 4B is another perspective view of headache-treatment device 100 that includes head piece 110, electrode cover 416, and spring electrodes 142, without the adjustable strap (for clarity of illustration), according to some embodiments of the invention.

FIG. 5A is perspective view of headache-treatment device 100 that includes head piece 110 with cover 416 removed to expose spring electrodes 142, according to some embodiments of the invention.

FIG. 5B is another perspective view of headache-treatment device 100 that includes head piece 110 with electrode cover 416 removed to expose spring electrodes 142, and gel pack 550, according to some embodiments of the invention.

FIG. 6A is perspective view of headache-treatment device 100 that includes head piece 110 with electrode cover 416 removed to insert gel pack 550, according to some embodiments of the invention.

FIG. 6B is another perspective view of headache-treatment device 100 that includes head piece 110 with electrode cover 416 and gel pack 550 in position to apply conductive gel to spring electrodes 142, according to some embodiments of the invention.

FIG. 7A is perspective view of headache-treatment device 100 that includes head piece 110 with electrode cover 416 (shown with gel pack 550 removed) and spring electrodes 142, according to some embodiments of the invention.

FIG. 7B is another perspective view of headache-treatment device 100 that includes head piece 110 and spring electrodes 142, according to some embodiments of the invention.

FIG. 8A is perspective view of headache-treatment device electrode subassembly 800 that includes electrode base 810, spring electrodes 142, electrode connecting busbars 820 and 830, and electrical connection wires 822 and 832, according to some embodiments.

FIG. 8B is a back perspective view of headache-treatment device electrode subassembly 800 that includes electrode base 810, spring electrodes 142, electrode connecting busbars 820 and 830, and wires 822 and 832, according to some embodiments of the invention.

FIG. 8C is a front perspective view of headache-treatment device electrode subassembly 800 that includes base 810, spring electrodes 142, according to some embodiments.

FIG. 8D is a top perspective view of headache-treatment device electrode subassembly 800 that includes electrode base 810, spring electrodes 142, electrode connecting busbars 820 and 830 (busbar 830 below busbar 820 is not visible), and connection wires 822 and 832 (wire 832 and portions of wire 822 are not visible), according to some embodiments.

FIG. 8E is a back-left-side perspective view of headache-treatment device electrode subassembly 800 (without spring electrodes 142) that includes electrode base 810, electrode connecting busbars 820 and 830, according to some embodiments of the invention.

FIG. 8F is a front-left-side perspective view of headache-treatment device electrode subassembly 800 (without spring electrodes 142) that includes electrode base 810, electrode connecting busbars 820 and 830, according to some embodiments of the invention.

FIG. 8G is a perspective exploded view of head piece 110 that includes spring electrodes 142, electrode base 810, and base housing 805, according to some embodiments.

FIG. 8H is a front-left-side perspective view of base housing 805, according to some embodiments of the invention.

FIG. 9A is a perspective view of spring electrodes 142, according to some embodiments of the invention.

FIG. 9B is a perspective view of spring electrodes 142 with cover 920 removed to illustrate sponge-like material 930, according to some embodiments of the invention.

FIG. 9C is a side view that illustrates positioning of spring electrodes 142 of a headache-treatment device in place on the occiput of patient 99 according to some embodiments.

FIG. 9D is a cross-sectional side view that illustrates the inner structure of a spring electrode 142 according to some embodiments of the invention.

FIG. 10A is a circuit block diagram of a system 1001 of some embodiments of the invention including signal generator 1010 and amplifier module 1015.

FIG. 10B is a circuit block diagram of a system 1002 of some embodiments of the invention including at least two signal generators, with signal generator 1010 and signal generator 1012, along with amplifier module 1017.

FIG. 10C is a pulse diagram 1003 of a 5 kHz square pulse stream S1 of some embodiments of the invention.

FIG. 10D is a pulse diagram 1004 of a 5.250 kHz square pulse stream S2 of some embodiments of the invention.

FIG. 10E is a pulse diagram 1005 of the summation stimulation signal of streams S1+S2 of some embodiments of the invention.

FIG. 10F is a FFT (fast Fourier transform) diagram 1006 of the summation of the S1 pulse stream of diagram 1003 and S2 pulse stream of some embodiments of the invention.

FIG. 11A is a front view of controller/power module 320, according to some embodiments of the invention.

FIG. 11B is a front-perspective view of controller/power module 320, according to some embodiments of the invention.

FIG. 11C is a front-perspective view of controller/power module 320 in the hand of operator 98, according to some embodiments of the invention.

FIG. 11D is a front-perspective view of controller/power module 320 that illustrates operator 98 pressing the power-on/off button 1110 of controller/power module 320, according to some embodiments of the invention.

FIG. 12A is a front-perspective view of controller/power module 320 that illustrates operator 98 about ready to press the start button 1220, according to some embodiments.

FIG. 12B is a front-perspective view of controller/power module 320 that illustrates operator 98 about ready to adjust the intensity button 1230, according to some embodiments.

FIG. 13A is a back-perspective view of controller/power module 320 that illustrates operator 98 about ready to release the latch 1320 on battery-compartment cover 1310, according to some embodiments of the invention.

FIG. 13B is a back-perspective view of controller/power module 320 that illustrates battery-compartment cover 1310 removed to provide access to batteries 1330, according to some embodiments of the invention.

FIG. 14A is a front-perspective view of controller/power module 320 that illustrates module body 1410 and internal electronics with front cover 1420 removed, according to some embodiments of the invention.

FIG. 14B is a back-perspective view of controller/power module 320 that illustrates battery-compartment cover 1310 removed to provide access to batteries 1330, according to some embodiments of the invention.

FIG. 14C is a back-perspective view of controller/power module body 1410, front cover 1420, and battery-compartment cover 1310, according to some embodiments.

FIG. 15A is a front-left-perspective view of a combined occipital-and-supraorbital transcutaneous nerve stimulator unit 1500, according to some embodiments of the invention.

FIG. 15B is a front-left-perspective view of combined occipital-and-supraorbital transcutaneous nerve stimulator unit 1500 in place on both the front and back of the head of patient 99, according to some embodiments of the invention.

FIG. 15C is a back-left-perspective view of combined occipital-and-supraorbital transcutaneous nerve stimulator unit 1500, according to some embodiments of the invention.

FIG. 15D is a side-perspective view of combined occipital-and-supraorbital transcutaneous nerve stimulator unit 1500 in place on both the front and back of the head of patient 99, according to some embodiments of the invention.

FIG. 15E is a back-perspective view of combined occipital-and-supraorbital transcutaneous nerve stimulator unit 1500, according to some embodiments of the invention.

FIG. 16A is a front-left-perspective view of a combined occipital-and-supraorbital transcutaneous nerve stimulator unit 1600 having a separate placement-holder 1649, according to some embodiments of the invention.

FIG. 16B is a front-left-perspective view of combined occipital-and-supraorbital transcutaneous nerve stimulator unit 1600 having a separate placement-holder 1649 holding the back portion 1640 in place on the back of the head of patient 99, according to some embodiments.

FIG. 16C is a top-perspective view of combined occipital-and-supraorbital transcutaneous nerve stimulator unit 1600 having a separate placement-holder 1649, according to some embodiments of the invention.

FIG. 16D is a side-perspective view of combined occipital-and-supraorbital transcutaneous nerve stimulator unit 1600 having a separate placement-holder 1649 with front unit 1641 and back unit 1640 both in place on both the front and back of the head of patient 99, according to some embodiments of the invention.

FIG. 16E is a front-left-perspective view of combined occipital-and-supraorbital transcutaneous nerve stimulator unit 1600 having a separate placement-holder 1649 holding the back portion 1640 in place on the back of the head of patient 99 with front unit 1641 resting on back unit 1640, according to some embodiments of the invention.

FIG. 16F is a front-left-perspective view of combined occipital-and-supraorbital transcutaneous nerve stimulator unit 1600 having a separate placement-holder 1649 holding the back portion 1640 in place on the back of the head of patient 99 with front unit 1641 shown in place in the front of the patient's head and in phantom lines in intermediate positions from resting on top of back unit 1640, according to some embodiments of the invention.

FIG. 17A is a top-perspective view of combined occipital-and-supraorbital-and-temporal transcutaneous nerve stimulator unit 1700 having additional side electrodes 1747 on either side of the front electrodes 1743 on front unit 1741, according to some embodiments.

FIG. 17B is a front-left-perspective view of combined occipital-and-supraorbital-and-temporal transcutaneous nerve stimulator unit 1700, according to some embodiments.

FIG. 17C is a front-left-perspective view of combined occipital-and-supraorbital-and-temporal transcutaneous nerve stimulator unit 1701 having a separate placement-holder 1749 and having additional side electrodes 1747 on either side of the front electrodes 1743 on front unit 1741, according to some embodiments of the invention.

FIG. 17D is a front-left-perspective view of combined occipital-and-supraorbital-and-temporal transcutaneous nerve stimulator unit 1700 having a separate placement-holder 1749 holding the back portion 1740 in place on the back of the head of patient 99 with front unit 1741 shown in place in the front of the patient's head, according to some embodiments.

FIG. 18 is a flow chart of a method 1800, according to some embodiments.

### DETAILED DESCRIPTION OF THE INVENTION

Although the following detailed description contains many specifics for the purpose of illustration, a person of ordinary skill in the art will appreciate that many variations and alterations to the following details are within the scope of the invention. Accordingly, the following preferred embodiments of the invention are set forth without any loss of generality to, and without imposing limitations upon the claimed invention. In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention.

The leading digit(s) of reference numbers appearing in the Figures generally corresponds to the Figure number in which that component is first introduced, such that the same reference number is used throughout to refer to an identical component which appears in multiple Figures. Signals and connections may be referred to by the same reference number or label, and the actual meaning will be clear from its use in the context of the description.

As used herein, an electrolyte is any substance containing free ions that make the substance electrically conductive. As used herein, a gel is defined as a substantially dilute cross-linked system, which exhibits little or no flow when in the steady-state. By weight, gels are mostly liquid, yet they behave somewhat like solids due to a three-dimensional cross-linked network within the liquid. As used herein an electrolyte gel is an electrically conductive gel. One example of an electrically conductive gel suitable for TENS applications is Lectron II Conductivity Gel by Pharmaceutical Innovations, Inc. Another example is Spectra 360® Electrode Gel by Parker Labs, Inc. Other conductive gels suitable for TENS applications are generally available and may be used as an electrolyte gel in various embodiments.

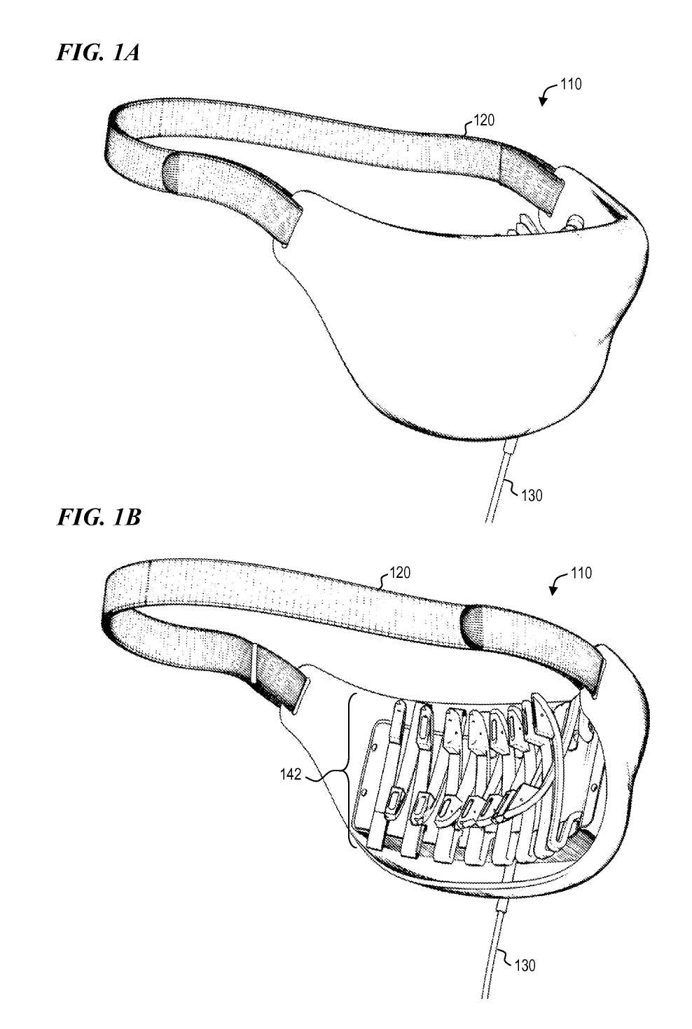

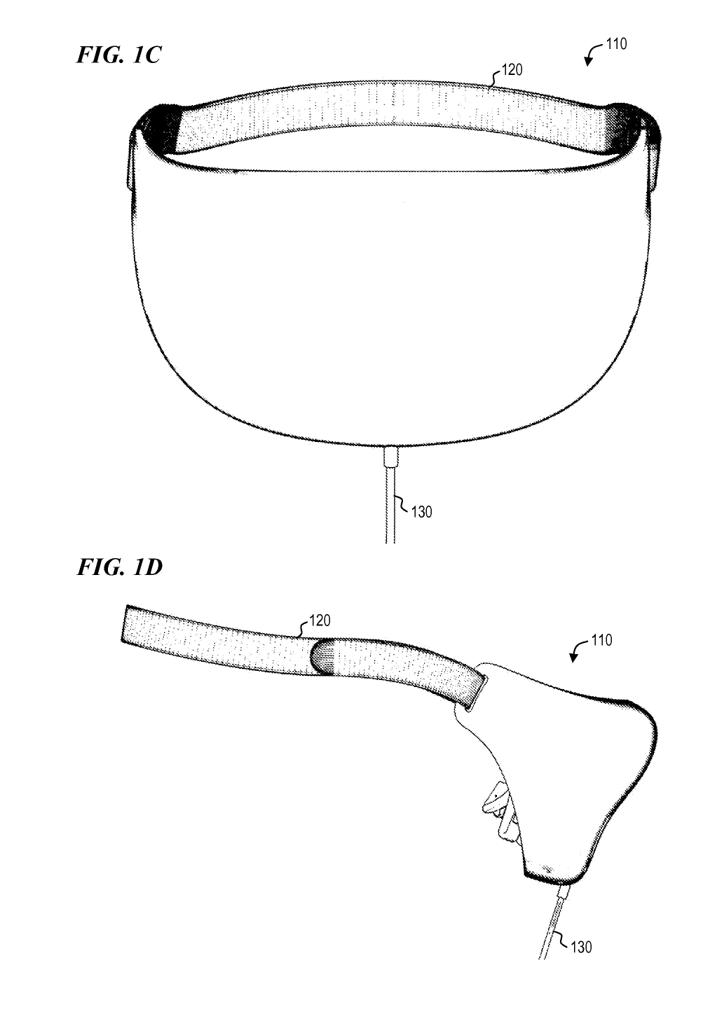

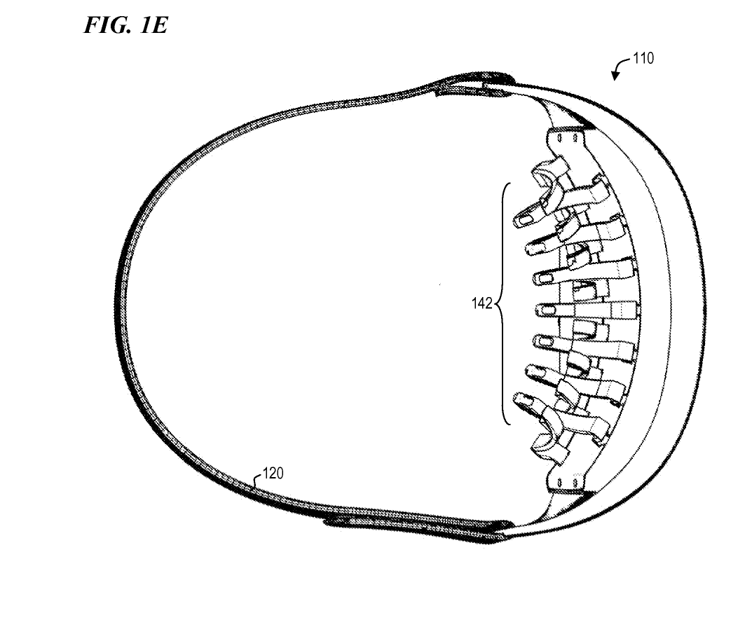

FIG. 1A is a back-left-side perspective view that illustrates a head piece 110 of a headache-treatment device according to some embodiments of the invention. In some embodiments, the head piece 110 includes an adjustable strap 120 for holding the head piece 110 in position on the patient. As shown, the head piece has a connecting electrical cable 130 to the control/power module. In some other embodiments, the control/power module is built into head piece 110. In some embodiments, strap 120 includes a hook-and-loop material (e.g., Velcro®-brand fasteners or the like) for attaching the end of the strap once it is placed through the slot opening at the end of head piece 110 and adjusted to a suitable length. In some embodiments, strap 120 includes at least a length of elastic to provide a comfortable and snug fit.

FIG. 1B is a front-left-side perspective view of head piece 110 (which is shown from a different viewpoint in FIG. 1A). From this view, the spring electrodes 142 used to make electrical connection to the scalp of the patient can be seen on the inside of headpiece 110.

FIG. 1C is a back perspective view of head piece 110 (which is shown from a different viewpoint in FIG. 1A) according to some embodiments of the invention.

FIG. 1D is a left-side perspective view of head piece 110 (which is shown from a different viewpoint in FIG. 1A) according to some embodiments of the invention.

FIG. 1E is a top perspective view of head piece 110 (which is shown from a different viewpoint in FIG. 1A) according to some embodiments of the invention.

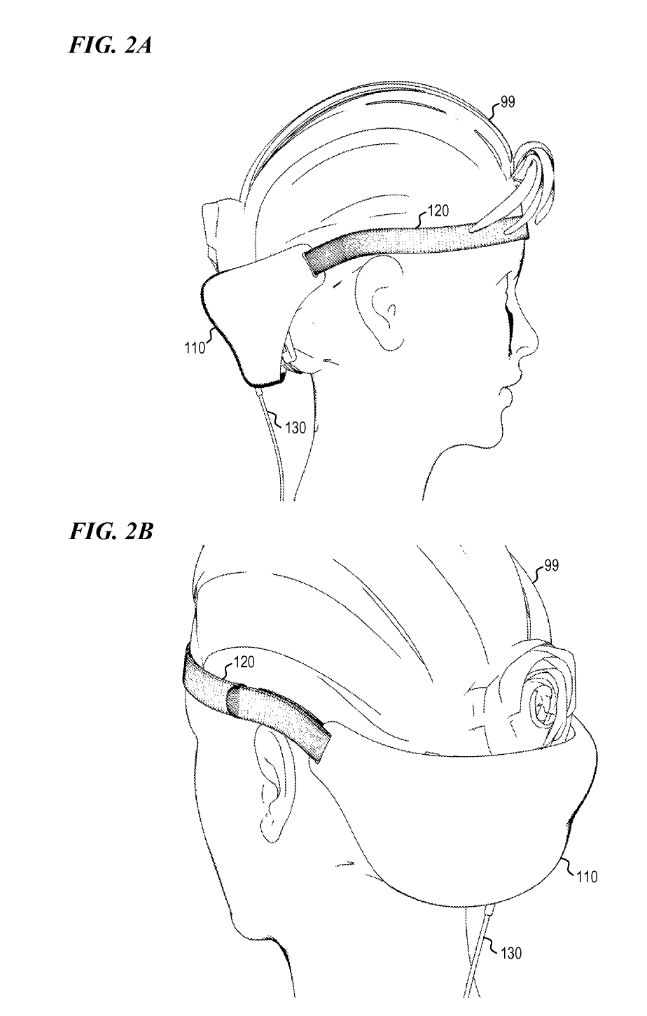

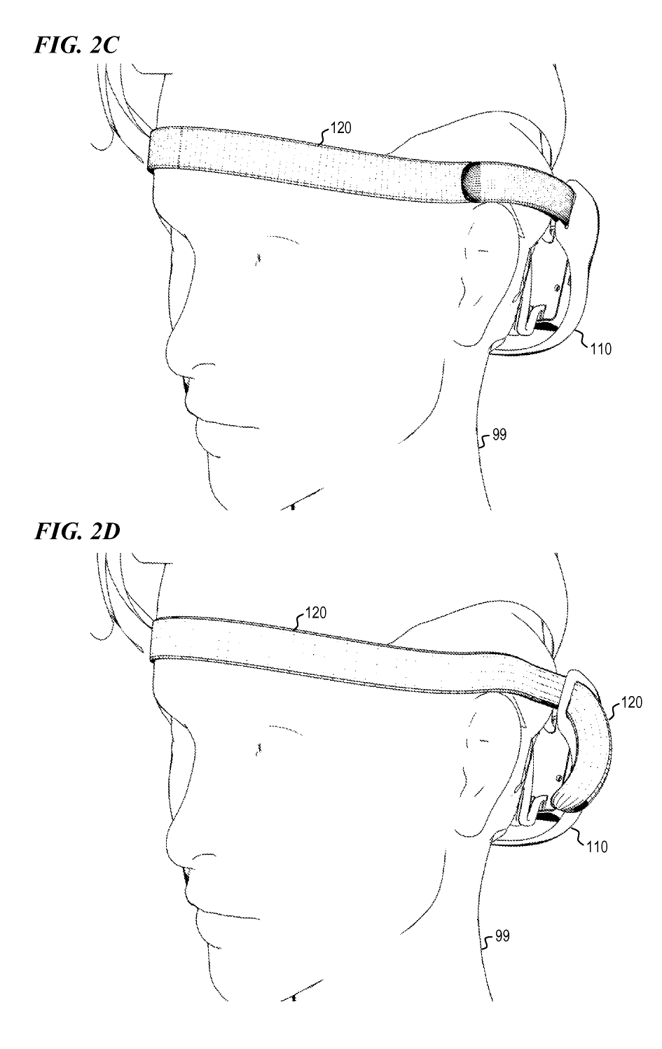

FIG. 2A is a right-side perspective view that illustrates head piece 110 of a headache-treatment device in place on the occiput (back of the head) of patient 99 according to some embodiments of the invention.

FIG. 2B is a back-left-side perspective view that illustrates head piece 110 of a headache-treatment device in place on the occiput of patient 99 according to some embodiments.

FIG. 2C is a front-left-side perspective view that illustrates head piece 110 of a headache-treatment device in place on the occiput of patient 99 according to some embodiments.

FIG. 2D is a front-left-side perspective view that illustrates head piece 110 of a headache-treatment device with adjustable head strap 120 loosened for adjustment according to some embodiments of the invention. In some embodiments, strap 120 includes a hook-and-loop material (e.g., Velcro®-brand fasteners or the like) for attaching the end of the strap once it is placed through the slot opening at the end of head piece 110 and adjusted to a suitable length, and in this view the hook-and-loop is not yet affixed in place (FIG. 2C shows the end after the hook-and-loop material is affixed). Adjustment to a suitable length of strap 120 includes adjusting for tension, comfort, stability in position on patient 99, electrical contact, and/or effectiveness of TNS.

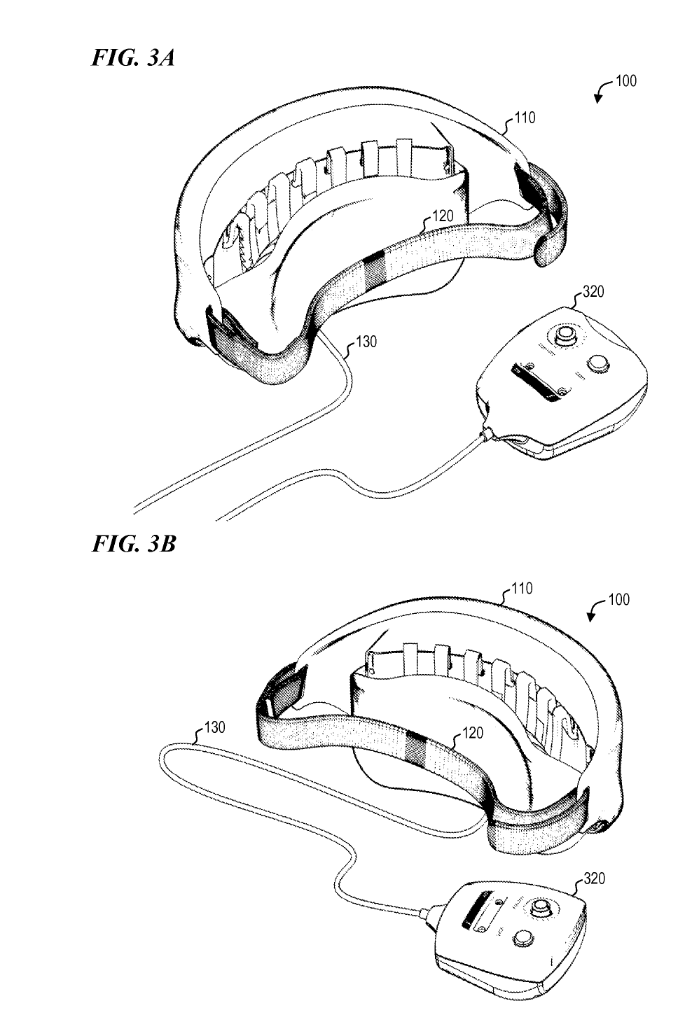

FIG. 3A is a perspective view of headache-treatment device 100 that includes head piece 110, controller/power module 320, and connecting electrical cable 130 according to some embodiments of the invention. FIG. 3B is another perspective view of headache-treatment device 100 that includes head piece 110, controller/power module 320, and connecting electrical cable 130 according to some embodiments of the invention.

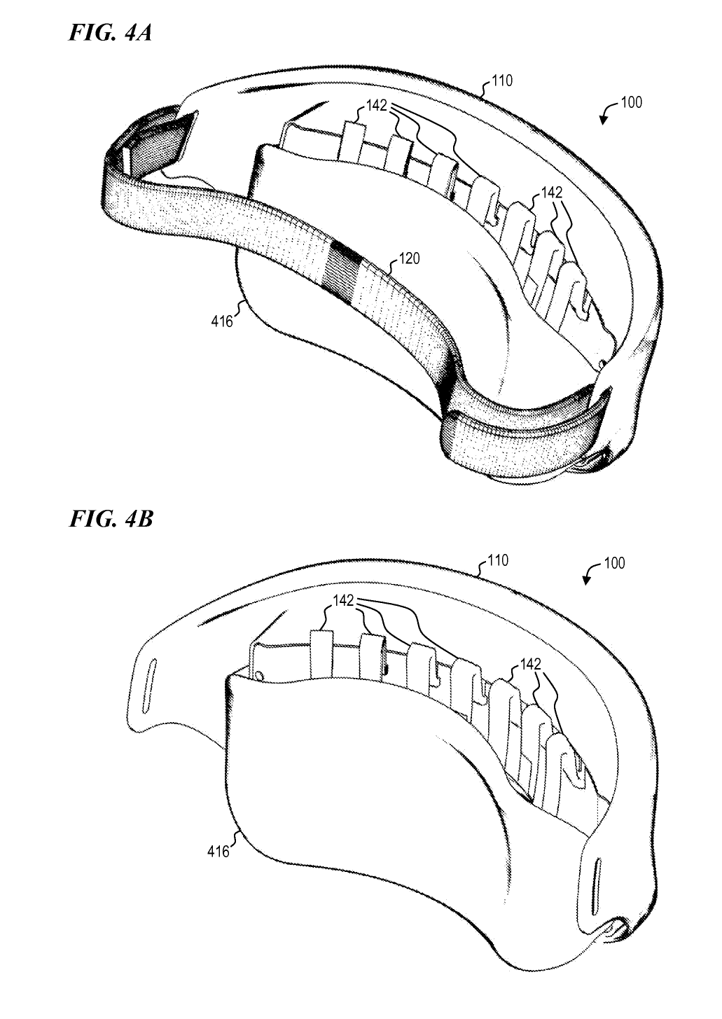

FIG. 4A is perspective view of headache-treatment device 100 that includes head piece 110, adjustable strap 120, electrode cover 416, and spring electrodes 142 according to some embodiments of the invention. FIG. 4B is another perspective view of headache-treatment device 100 that includes head piece 110, electrode cover 416, and spring electrodes 142, without the adjustable strap (for clarity of illustration), according to some embodiments of the invention.

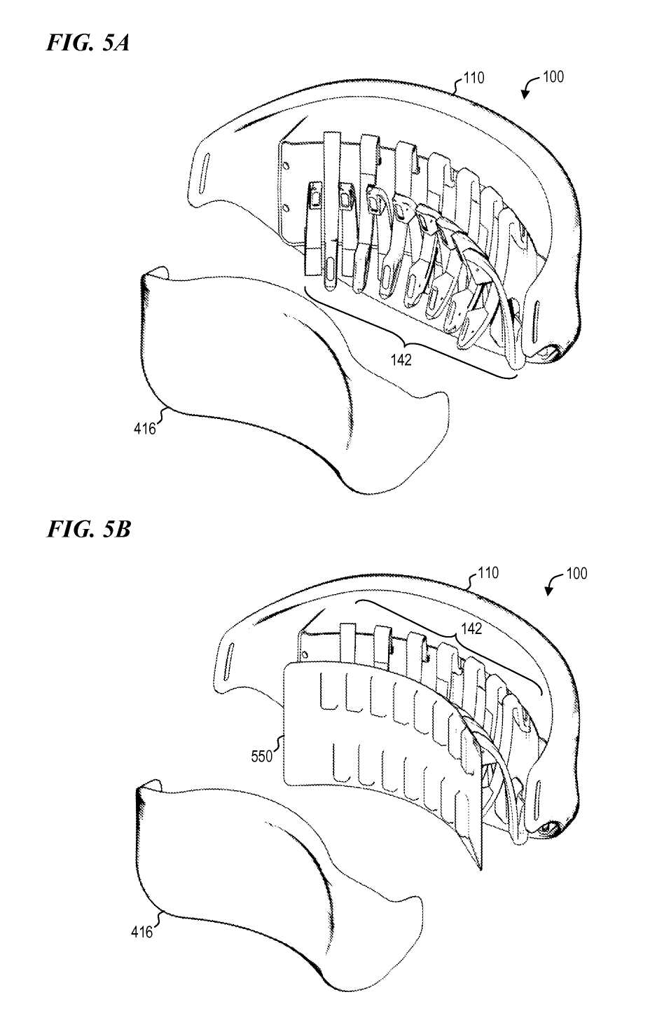

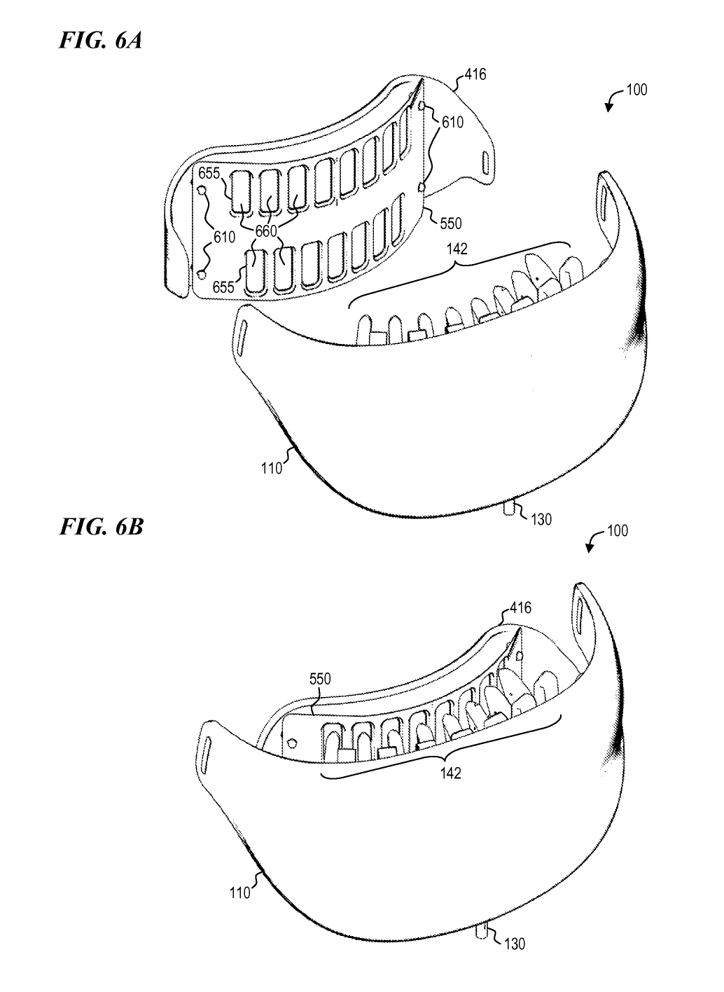

FIG. 5A is perspective view of headache-treatment device 100 that includes head piece 110 with electrode cover 416 removed to expose spring electrodes 142, according to some embodiments of the invention. In some embodiments, electrode cover 416 is molded from plastic or a polymer. In some embodiments, the electrode cover 416 can be used to press the gel pack 550 (shown in FIG. 5B and FIGS. 6A-6B) against the electrodes in order to puncture the foil (or polymer film, or metalized polymer film) covering over the gel-filled pockets and thus apply the gel substantially simultaneously to a plurality of the electrodes. In some embodiments, the electrode cover 416 has a smaller radius of curvature than the radius of curvature of the row of electrodes, in order that electrode cover 416 can be “rolled” across the gel pack and/or the gel pack 550 can be “rolled” across the electrodes, to sequentially puncture fewer than all the gel pockets at a time while quickly puncturing all of the desired pockets that correspond to the number of electrodes, in order to reduce the amount of force needed to puncture the foil cover of the gel pack 550.

FIG. 5B is another perspective view of headache-treatment device 100 that includes head piece 110 with electrode cover 416 removed to expose spring electrodes 142, and gel pack 550, according to some embodiments of the invention.

FIG. 6A is perspective view of headache-treatment device 100 that includes head piece 110 with electrode cover 416 removed to attach gel pack 550, according to some embodiments of the invention. In some embodiments, Gel pack 550 is held in place on electrode cover 416 with retention tabs 610. Gel pack 550 includes a number of “blisters” or pockets 655, convex in shape away from the spring electrodes 142. In some embodiments, there is one pocket 655 corresponding to each spring electrode 142. In some embodiments, there is one pocket 655 corresponding to each pair of spring electrodes 142. In some other embodiments, there is one pocket 655 corresponding to some other combination of spring electrodes 142.

In some embodiments, in each pocket 655 (the side farthest from the spring electrodes 142) is an amount of electrolyte in the form of a conductive gel 660. The electrolyte in the form of a gel provides an effective electrolyte wetting agent for the electrodes and sponge-like tips of the electrodes, without the messy situation that could result with a straight liquid electrolyte wetting agent. One example of a conductive gel suitable for TENS applications is Lectron II Conductivity Gel by Pharmaceutical Innovations, Inc. Another example is Spectra 360® Electrode Gel by Parker Labs, Inc. In other embodiments, other conductive gels suitable for TENS applications are generally available and may be used as an electrolyte gel.

FIG. 6B is another perspective view of headache-treatment device 100 that includes head piece 110 with electrode cover 416 and gel pack 550 in position to apply conductive gel to spring electrodes 142, according to some embodiments of the invention. In some embodiments, when electrode cover 416 is moved into position with gel pack 550 in place (i.e., with the electrode cover 416 used to press, urge, and/or squeeze the gel pack 550 against the electrodes), electrodes 142 puncture the seal on gel pack 550 over each “blister.” In some embodiments, the seal is a metal foil. In some embodiments, the seal includes a metal foil (e.g., in combination with a paper or polymer-film layer), or a metalized polymer film. In other some embodiments, the seal is a peelable layer removed after the Gel pack 550 is held in place on electrode cover 416 with retention tabs 610. In yet other some embodiments, the seal is a metal foil with a protective peelable layer that is removed after the Gel pack 550 is held in place on electrode cover 416 with retention tabs 610. The protective peelable layer helps prevent a delicate foil layers from being punctured until ruptured by the spring electrodes 142.

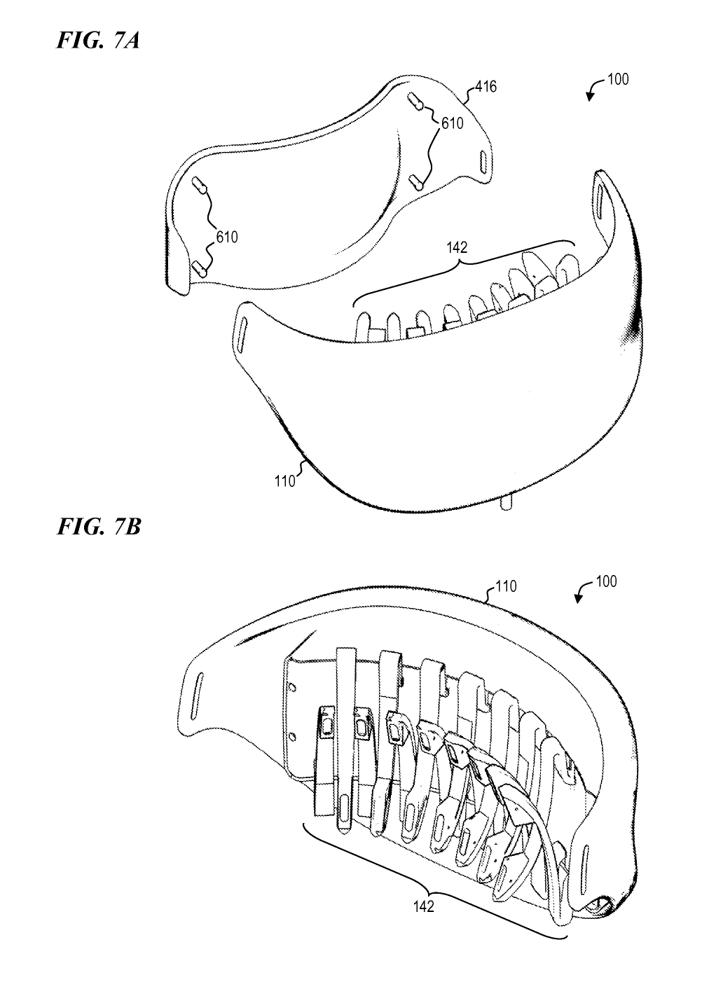

FIG. 7A is perspective view of headache-treatment device 100 that includes head piece 110 with electrode cover 416 (shown with gel pack 550 removed) and spring electrodes 142 and FIG. 7B is another perspective view of headache-treatment device 100 that includes head piece 110 and spring electrodes 142, according to some embodiments.

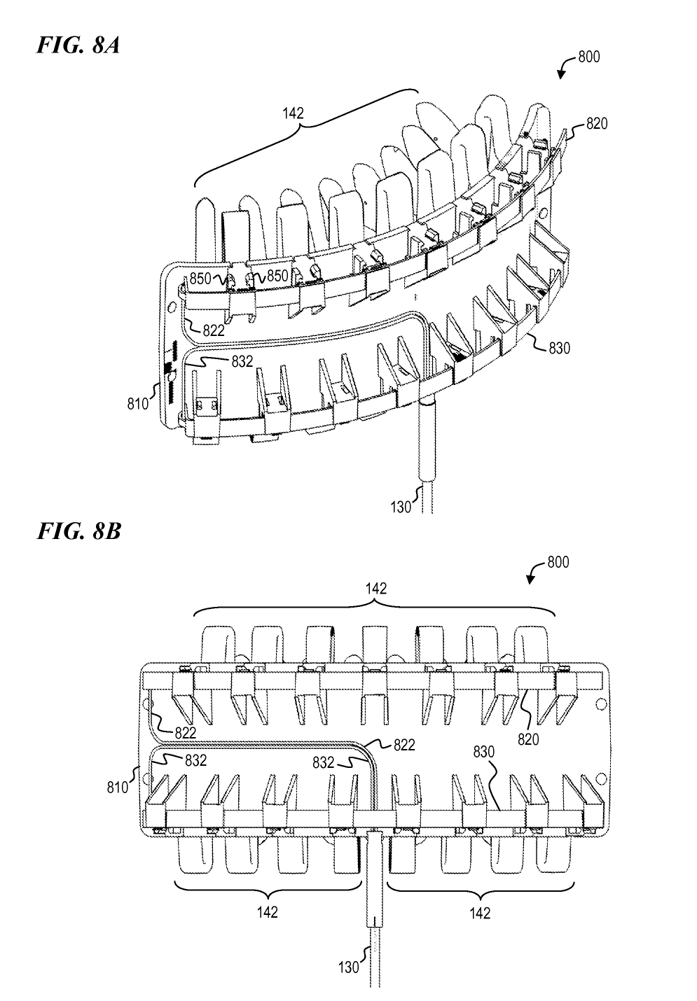

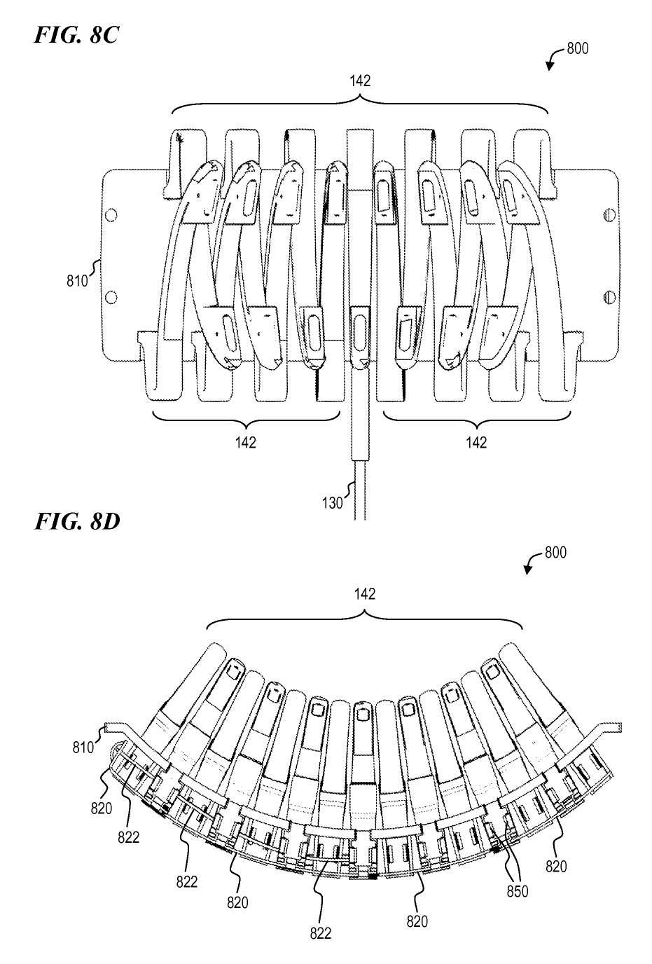

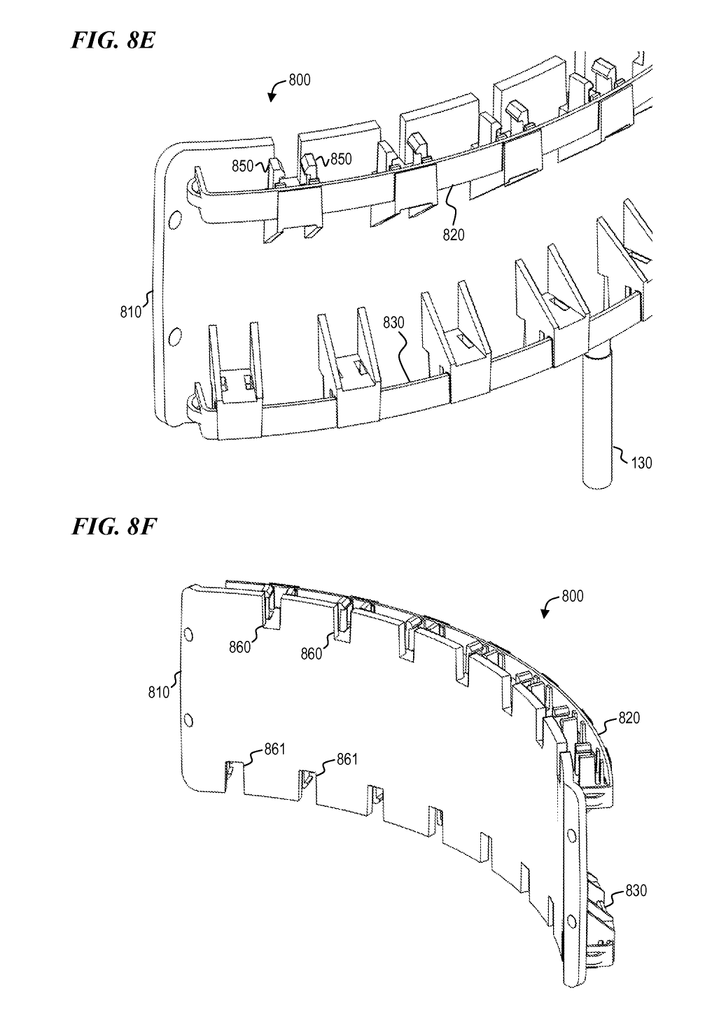

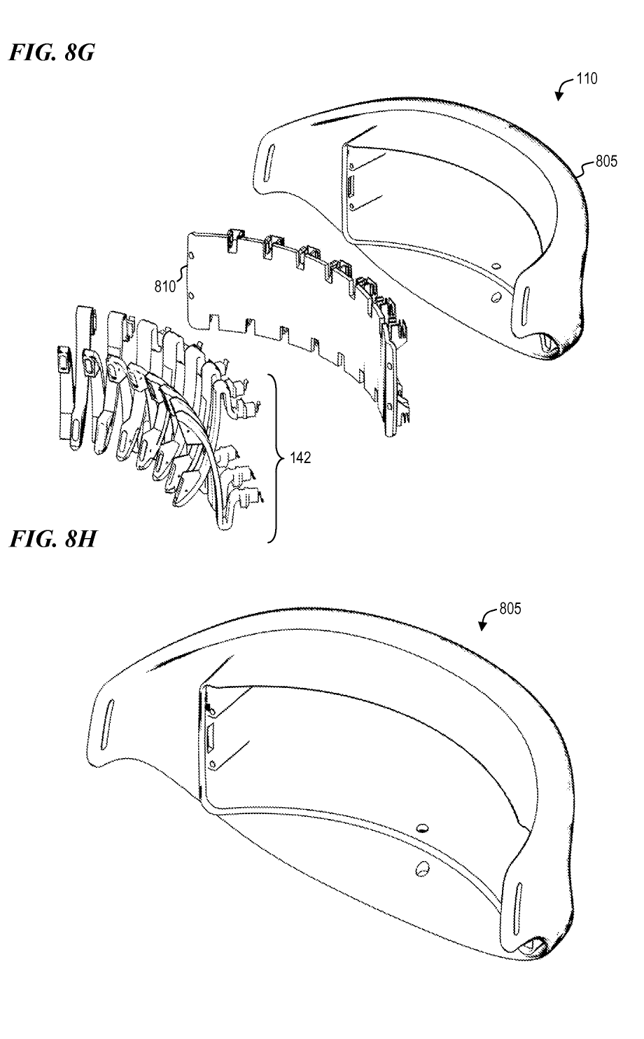

FIG. 8A is perspective view of headache-treatment device electrode subassembly 800 that includes electrode base 810, spring electrodes 142, electrode connecting busbars 820 and 830, and electrical connection wires 822 and 832, according to some embodiments of the invention. In some embodiments, electrode base 810 is molded from plastic or a polymer. In this embodiment, busbar 820 electrically connects wire 822 to the top row of spring electrodes 142, and busbar 830 electrically connects wire 832 to the bottom row of spring electrodes 142. Electrical connection wires 822 and 832 connect to controller/power module 320 through connecting electrical cable 130. Connection wires 822 supplies one pole of the TENS output and 832 supplies the other pole of the TENS output. Electrode base 810 includes a pair of retention clips 850 for each spring electrodes 142 to secure the spring electrodes in position, while also allowing them to be removed for replacement if necessary.

FIG. 8B is a back perspective view of headache-treatment device electrode subassembly 800 that includes electrode base 810, spring electrodes 142, electrode connecting busbars 820 and 830, and electrical connection wires 822 and 832, according to some embodiments of the invention. FIG. 8C is a front perspective view of headache-treatment device electrode subassembly 800 that includes electrode base 810, spring electrodes 142, according to some embodiments of the invention. FIG. 8D is a top perspective view of headache-treatment device electrode subassembly 800 that includes electrode base 810, spring electrodes 142, electrode connecting busbars 820 and 830 (busbar 830 below busbar 820 is not visible in this view), and electrical connection wires 822 and 832 (wire 832 and portions of wire 822 are not visible), according to some embodiments of the invention. FIG. 8E is a back-left-side perspective view of headache-treatment device electrode subassembly 800 (without spring electrodes 142) that includes electrode base 810, electrode connecting busbars 820 and 830 (without electrical connection wires 822 and 832), according to some embodiments.

FIG. 8F is a front-left-side perspective view of headache-treatment device electrode subassembly 800 (without spring electrodes 142) that includes electrode base 810, electrode connecting busbars 820 and 830, according to some embodiments of the invention. In some embodiments, busbars 820 and 830 are formed from stainless steel, copper, or some conductive material. In some embodiments, such as the one illustrated, electrode base 810 includes seven upper slots 860 for spring electrodes 142 interleaved between eight lower slots 861 for spring electrodes 142. In other embodiments, other numbers of upper/lower electrodes may be used.

FIG. 8G is a perspective exploded view of head piece 110 that includes spring electrodes 142, electrode base 810, and base housing 805, according to some embodiments.

FIG. 8H is a front-left-side perspective view of base housing 805, according to some embodiments of the invention. In some embodiments, base housing 805 is molded from a plastic such as a polymer or a ceramic, or the like.

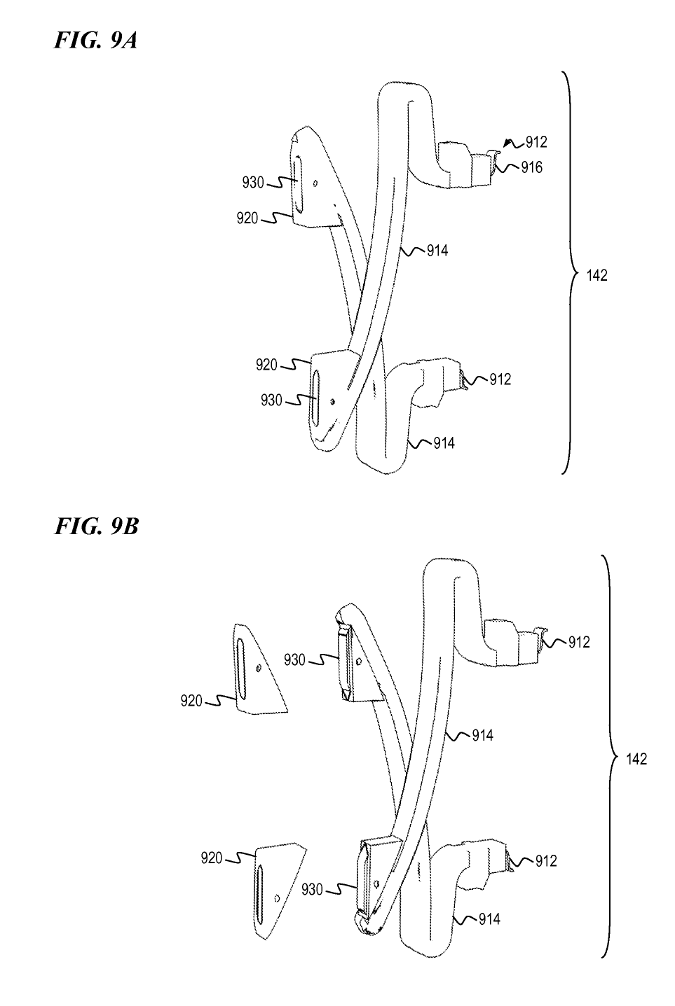

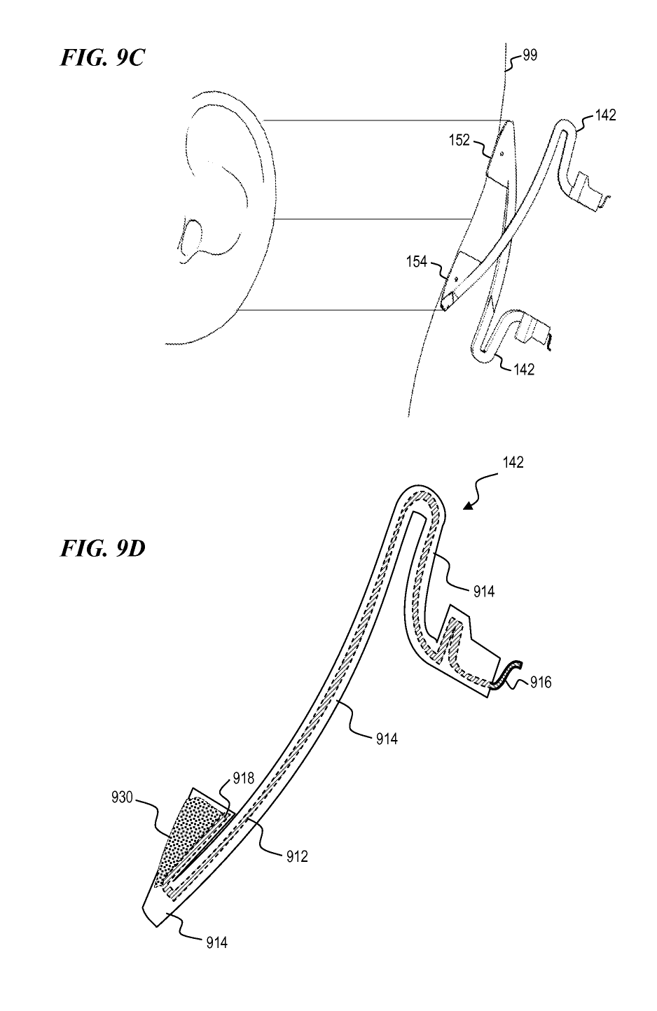

FIG. 9A is a perspective view of spring electrodes 142, according to some embodiments of the invention. In some embodiments, spring electrodes 142 include an inner spring stainless steel base 912 bent into the desired shape. The proximal end 916 of stainless steel base 912 is shaped to make electrical contact with busbars 820 and 830 when spring electrodes 142 are inserted into in electrode base 810. In some embodiments, the spring electrodes 142 are shaped and bent to distribute bending stress such that the spring stainless steel base 912 resists breakage. In some embodiments, covering the spring base 912 is a plastic or polymer insulating cover 914 covering all but the ends. The plastic or polymer covering provides the desired shape to fit in to the upper slots 860 and lower slots 861 in electrode base 810 in addition to providing electrical insulation. The patient-contacting ends of spring electrodes 142 includes an electrically conductive sponge or sponge-like material 930 to absorb the conductive gel and to make effective electrical contact to the scalp of the patient, in some cases, contact through hair. The distal end 918 (see FIG. 9D) of the spring stainless steel base 912 is in electrical contact to the sponge-like material 930. Covering some of the sponge or sponge-like material 930 and holding it in place is the cover 920. In some embodiments, end cover 920 is a rubber or rubber-like material attached to insulating cover 914 to provide a comfortable contact with the patient. In some embodiments, a conductive mesh or textile, such as described in U.S. Pat. No. 5,374,283 to Flick, which is incorporated herein by reference, is used in place of or in addition to the sponge-like material 930.

FIG. 9B is a perspective view of spring electrodes 142 with end cover 920 removed from insulating cover 914 to illustrate sponge-like material 930, according to some embodiments of the invention. In some embodiments, end cover 920 is removable to allow replacement of conductive sponge or sponge-like material 930 and/or end cover 920.

FIG. 9C is a side view that illustrates positioning of spring electrodes 142 of a headache-treatment device in place on the occiput of patient 99 according to some embodiments. In some embodiments, at least some of the spring electrodes 142 contact the user or patient in a cranial position 152 of the occipital region of the back of the head, and at least some of the spring electrodes 142 contact the user or patient in a caudal position 154 of the occipital region of the back of the head. In some embodiments, transcutaneous electrical stimulation between the cranial contact 152 (cranial being the superior, or upper-on-the-head, contact) and the caudal contact 154 (caudal being the inferior, or lower-on-the-head, contact relative to the cranial contact) induces an occipital region nerve response.

FIG. 9D is a cross-sectional side view that illustrates the inner structure of a spring electrode 142 according to some embodiments. The proximal end 916 of stainless steel base 912 is shaped to make electrical contact with busbars 820 and 830 (see FIG. 8A) when spring electrodes 142 are inserted into in electrode base 810. In some embodiments, an electrically insulating polymer cover 914 covers the spring base 912 over all but the proximal and distal ends. The distal end 918 of the spring stainless steel base 912 is in electrical contact with the sponge-like material 930. In some embodiments, the spring electrode 142 in FIG. 9D is inverted or used in other orientations such as a caudal contact 154 in FIG. 9C. Spring contacts 142 are additionally used in some embodiments for supraorbital and/or temporal contacts for transcutaneous nerve stimulation (further described below).

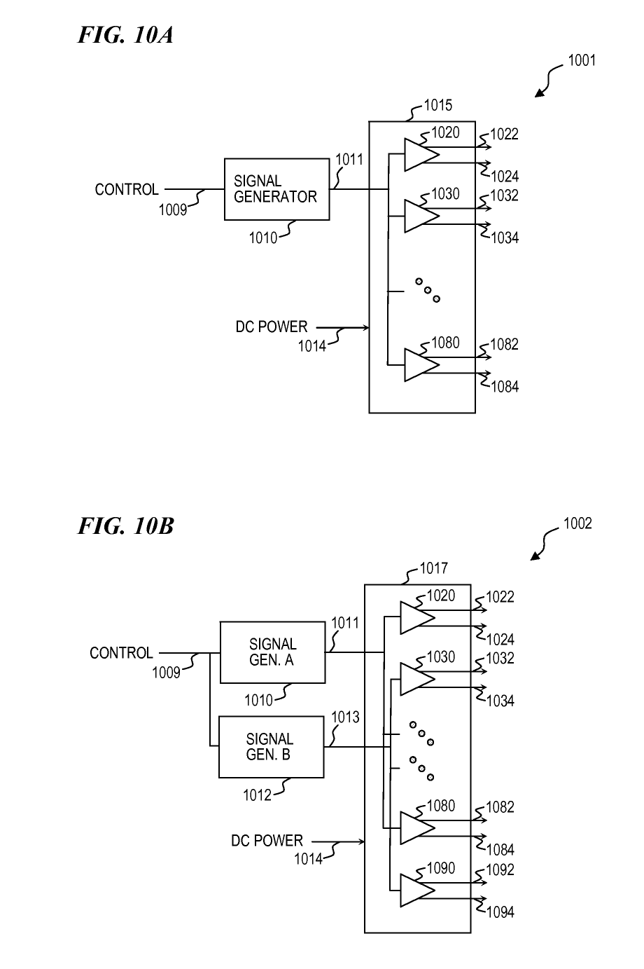

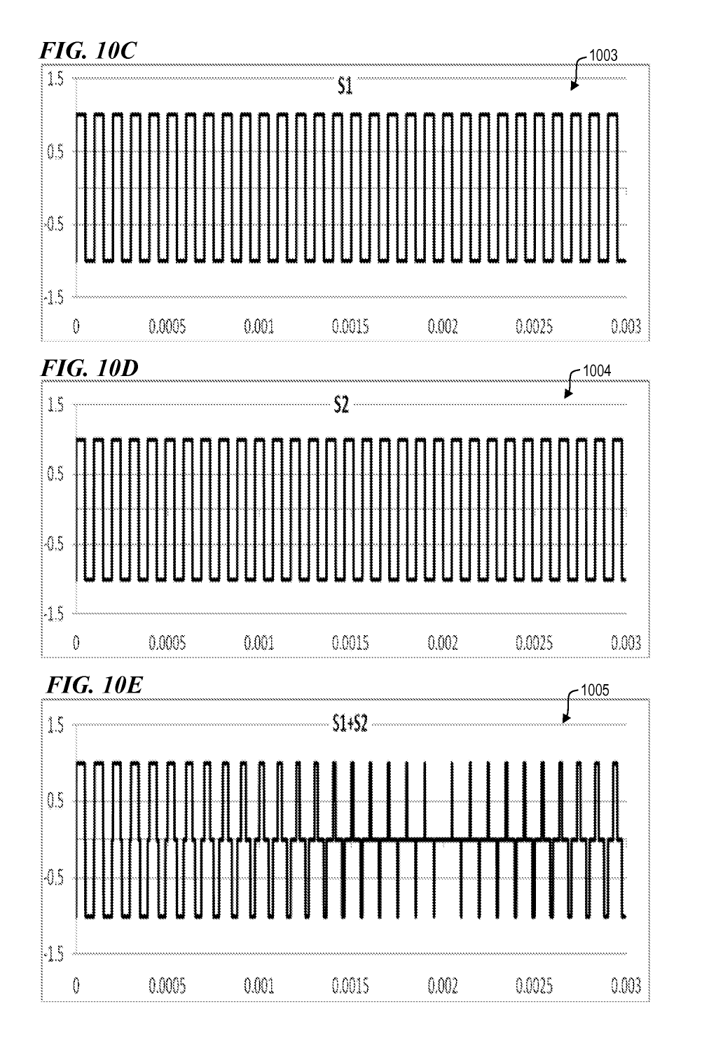

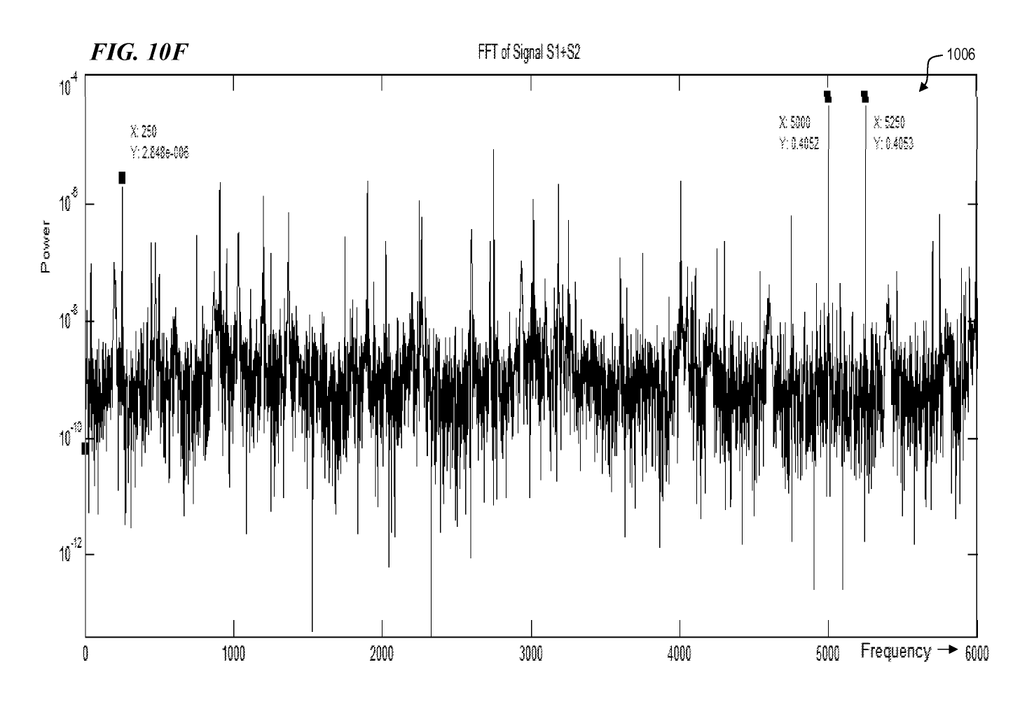

FIG. 10A is a circuit block diagram of a system 1001 of some embodiments of the invention. Circuit block diagram 1001 includes signal generator 1010 and amplifier module 1015. One or more control lines 1009 connect to, and control, signal generator 1010. In some embodiments, the one or more control lines 1009 have digital information (e.g., pulses or binary data streams) that are interpreted by, and/or control operation of, signal generator 1010 to control one or more aspects of the signal generator output 1011. In some embodiments, control lines 1009 have digital information to control one or more of the frequency, pulse rate, and amplitude (including the magnitude and/or whether the output is to be monophasic or biphasic) of the signal generator output signal(s) 1011. In some embodiments, amplifier module 1015 is contained within head piece 110 and signal generator 1010 is contained within controller/power module 320. In other embodiments, both are contained in the controller/power module 320, while in yet other embodiments, both are contained in the within head piece 110. In some embodiments, amplifier module 1015 includes buffer amplifiers 1020, 1030, . . . 1080 which amplify the signal generator output signal(s) 1011 and drive the respective differential output pairs 1022-1024, 1032-1034, . . . , and 1082-1084, as shown in FIG. 10A. In some embodiments, each respective differential pair is electrically isolated from the others of the differential output pairs. In some embodiments, each pair of differential outputs (e.g., differential outputs 1022 and 1024 from buffer amplifier 1020) are operatively connected to one pair, or to a plurality of pairs, of spring electrodes 142. In some embodiments, one of the differential outputs (e.g., differential output 1022) is operatively connected to one or more of the cranial row (top row) of spring electrodes 142 of headpiece 110 and the other differential output (e.g., differential output 1024) is operatively connected to a corresponding one or more of the caudal row (bottom row) spring electrodes 142 of headpiece 110. In some embodiments, one of each pair (e.g., caudal electrodes 1024, 1034, through 1084) of the plurality of pairs of electrodes is a ground or common electrode, and the other electrode of the pair is driven by respective single-ended buffer amplifiers 1020-1080 with a biphasic (or monophasic) pulse stream relative to that ground. This configuration provides longitudinal (i.e., lengthwise along a nerve) transcutaneous electrical stimulation to the target nerves when the nerves of interest are in the caudal-cranial direction. Longitudinal transcutaneous electrical nerve stimulation is believed to be more effective than transverse electrical nerve stimulation. Less effective transverse electrical nerve stimulation includes a pair of electrodes with one electrode lateral on each side of the nerve, or one electrode on the skin near the nerve to be stimulated and another electrode or ground electrode elsewhere on the user, for example in the user's hand or attached to the user's wrist, ankle, or thigh. DC power input 1014 provides power to amplifier module 1015 and the buffer amplifiers.

In some embodiments, the buffer amplifier differential output pairs 1022-1024, 1032-1034, . . . and 1082-1084 are separately current and/or voltage limited. In embodiments that use a single current-limited buffer amplifier connected to a plurality of pairs of electrodes, if one of the electrodes does not touch or have good electrical contact to the patient's skin, more than the desired amount of current will flow through the other electrodes that are connected to the skin (e.g., if a single buffer amplifier is designed to limit to 60 mA and is connected to three pairs of electrodes with the expectation that each electrode pair would conduct 20 mA, the patient could receive all 60 mA through a single electrode pair if two electrodes were not contacting the skin. Current limiting each buffer amplifier's output(s) and connecting each buffer amplifier to a single pair of electrodes helps eliminate excess current if not all electrodes are in good electrical contact with the skin. Voltage limiting helps avoid arcing and/or burning when an electrode does not have good electrical contact with the patient's scalp. Having multiple independent pairs of electrodes and multiple respective buffer amplifiers with voltage and/or current limiting provides effective transcutaneous electrical stimulation even when one or more of the electrodes do not have good (i.e., low-impedance) electrical contact with the patient.

FIG. 10B is a circuit block diagram of a system 1002 of some embodiments of the invention. System 1002 is much the same as system 1001 just described, except that system 1002 includes a plurality of signal generators (e.g., two are shown in FIG. 10B), including signal generator 1010 and signal generator 1012, along with amplifier module 1017. DC power input 1014 provides power to amplifier module 1017 and the buffer amplifiers 1020, 1030, . . . 1080, and 1090. One or more control lines 1009 have digital information (e.g., pulses or binary data streams) that are interpreted by, and/or control operation of, the two signal generators 1010 and 1012. In some embodiments, control lines 1009 control one or more aspects of the signal generator output signal(s) 1011 and signal generator output signal(s) 1013. In some embodiments, control lines 1009 have digital information to control one or more of the frequency, pulse rate, and amplitude (including the magnitude and/or whether the output is to be monophasic (i.e., pulse streams having a plurality of successive pulses all having the same polarity) or biphasic (e.g., pulse streams having alternating polarities, wherein the charge delivered by each positive pulse is neutralized by one or more negative pulses in order to avoid local buildup of charge which can be detrimental to the patient)) of the signal generator output signal(s) 1011 independently and/or separately from those characteristics of the signal generator output signal(s) 1013. In some embodiments, such as shown in FIG. 10B, amplifier module 1017 includes pairs a plurality of sets of buffer amplifiers each with differential outputs, as shown in FIG. 10B, with one of each pair of buffer amplifiers operatively connected to signal generator output signal(s) 1011 and each of the other of each pair of buffer amplifiers operatively connected to signal generator output signal(s) 1013. In the example shown in FIG. 10B, buffer amplifiers 1020 and 1030 are members of one pair provided to drive differential outputs 1022-1024 and 1032-1034, respectively, while buffer amplifiers 1080 and 1090 are members of another pair that are provided to drive differential outputs 1082-1084, and 1092-1094, respectively, and zero or more other pairs of buffer amplifiers are provided to drive other differential outputs. In some embodiments, each buffer pair of differential outputs are operatively connected to adjacent pairs of spring electrodes 142 (e.g., one spring electrode 142 in the cranial row is operatively connected to differential amplifier output 1022, an adjacent spring electrode 142 in the cranial row is operatively connected to differential amplifier output 1032, and one spring electrode 142 in the caudal row is operatively connected to differential amplifier output 1024, and an adjacent spring electrode 142 in the caudal row is operatively connected to differential amplifier output 1034). This configuration provides longitudinal (i.e., lengthwise along a nerve) transcutaneous electrical stimulation to the target nerves with adjacent pairs of spring electrodes driven by different buffer amplifiers.

In some embodiments, each signal generator 1010 and 1012 drive the respective buffer amplifiers at a different pulse frequency. In some embodiments, each signal generator 1010 and 1012 drive the respective buffer amplifiers at a slightly different pulse frequency. In some embodiments, signal generator A 1010 drives the buffer amplifiers operatively connected to it at a first pulse rate of 5000 pps (pulses per second) and signal generator B 1010 drives the buffer amplifiers operatively connected to it at a second pulse rate of 5150 pps. The individual pulse streams of 5000 pps and 5150 pps have frequency components of 5000 Hz and 5150 Hz along with Fourier components at higher frequencies, and any amplitude modulation between the two signals yields a frequency sum of the differential outputs to the spring electrodes of 10150 pps (5150 pps plus 5000 pps), which transmits through skin capacitance more effectively than a low frequency because the skin has a lower impedance at higher frequencies. Thus the skin “sees” a high frequency and has a low impedance to those signals. The frequency as seen by nerves by the transcutaneous stimulation is the difference of the first and second frequencies, that is, 150 pps (5150 pps minus 5000 pps), which, in some embodiments, is effective for stimulation of nerve axons without stimulation of undesired muscle contractions. In other embodiments, the first frequency is in a range of about 1,000 to about 2,000 pps, a range of about 2,000 to about 3,000 pps, a range of about 3,000 to about 4,000 pps, a range of about 4,000 to about 6,000 pps, a range of about 6,000 to about 8,000 pps, a range of about 8,000 to about 10,000 pps, a range of about 10,000 to about 20,000 pps, a range of about 20,000 to about 40,000 pps, a range of about 40,000 to about 70,000 pps, a range of about 70,000 to about 100,000 pps, or a range above about 100,000 pps, and the second frequency is about 150 pps higher than the first frequency. In other embodiments, the second frequency is in a range of 60 to 80 pps higher than the first frequency, a range of 80 to 100 pps higher than the first frequency, a range of 100 to 120 pps higher than the first frequency, a range of 120 to 140 pps higher than the first frequency, a range of 140 to 160 pps higher than the first frequency, a range of 160 to 180 pps higher than the first frequency, a range of 180 to 200 pps higher than the first frequency, a range of 200 to 250 pps higher than the first frequency, or a range of more than 250 pps higher than the first frequency. Thus, in some embodiments, the two sets of pulse streams provide Fourier-frequency components, and/or modulate one another to produce sum-frequency components, that provide low-impedance connectivity to the skin of the patient, and difference-frequency components that provide effective nerve stimulation of targeted nerves without muscle stimulation. In some embodiments other frequencies and frequencies differences are used. In some embodiments, one or both sets of buffer amplifiers generate pulse streams that, over time, increase and decrease pulse widths to provide high-frequency signal components that reduce the impedance of the skin connections and low-frequency signal components that provide effective nerve stimulation of targeted nerves without muscle stimulation.

In some embodiments, the buffer amplifier differential outputs 1022-1024, 1032-1034, . . . , 1082-1084, and 1092-1094 are current and/or voltage limited, as set forth above in the description of current and/or voltage limiting for FIG. 10A. In some embodiments, the pulse streams from each pair of electrodes are not isolated and share a common ground. In some such embodiments, the electrodes have a sensing element such that the current at each electrode is monitored for a small amount of time after the onset of the pulse, and if no current is detected at any given electrode, then that electrode and its corresponding pair are disable (open circuit) for the duration of the pulse, or the pulse and its corresponding opposite phase. In some embodiments, all electrodes become active before each pulse or at the first phase of a pair of biphasic pulses.

Most, if not all, human tissue has parallel resistive and capacitive components. Given that the impedance due to the capacitive components decreases as the frequency of the excitation increases, a high-frequency pulse stream (e.g., in the kHz pulse-repetition-rate range) can be used to reduce the overall impedance of the skin and other non-excitable tissues. A reduction in impedance allows the current density to be higher farther away from the source (i.e., deeper into the tissue from the skin). Current density at the target tissue (e.g., a particular nerve) needs to be high enough to trigger a response; therefore, a high-frequency pulse stream results in a higher current density in the tissue at any given distance from the source. However, nerves do not respond to such high frequency pulses; therefore, in some embodiments such is described above for System 1002 of FIG. 10B, two or more high frequency pluses with a small frequency difference (1-500 Hz) are superimposed through simultaneous stimulation, through multiple electrodes, to generate an excitation signal at a lower frequency than any of the high frequency superimposed pulse streams. In some embodiments, a 5 kHz square pulse stream S1 (as shown is pulse diagram 1003 of FIG. 10C) and a 5.250 kHz square pulse stream S2 (as shown is pulse diagram 1004 of FIG. 10D), the summation stimulation signal of stream S1 plus stream S2 (also termed signal S1+S2) is created (as shown is pulse diagram 1005 of FIG. 10E) with higher power peaks than at the original frequencies of S1 and S2. FIG. 10F shows a FFT (fast Fourier transform) diagram 1006 of the summation of the S1 pulse stream of diagram 1003 and S2 pulse stream of diagram 1004, according to some embodiments. The FFT shows power peaks at the S1 pulse frequency of 5 kHz (i.e., pulse-repetition-rate of 5,000 pulses per second), the S2 pulse frequency of 5.250 kHz (i.e., pulse-repetition-rate of 5,250 pulses per second), and the Fourier transform of the summation signal includes a difference frequency of 250 Hz.

In other embodiments, the S1 pulse frequency is 5 kHz (i.e., pulse-repetition-rate of 5,000 pulses per second), the S2 pulse frequency is 5.150 kHz (i.e., pulse-repetition-rate of 5,150 pulses per second), and the Fourier transform of the summation includes a difference frequency of 150 Hz. In still other embodiments, other S1 and S2 pulse repetition rates are used to get similar difference frequencies. In yet other embodiments, other S1 and S2 pulse repetition rates are used to get yet other difference frequencies.

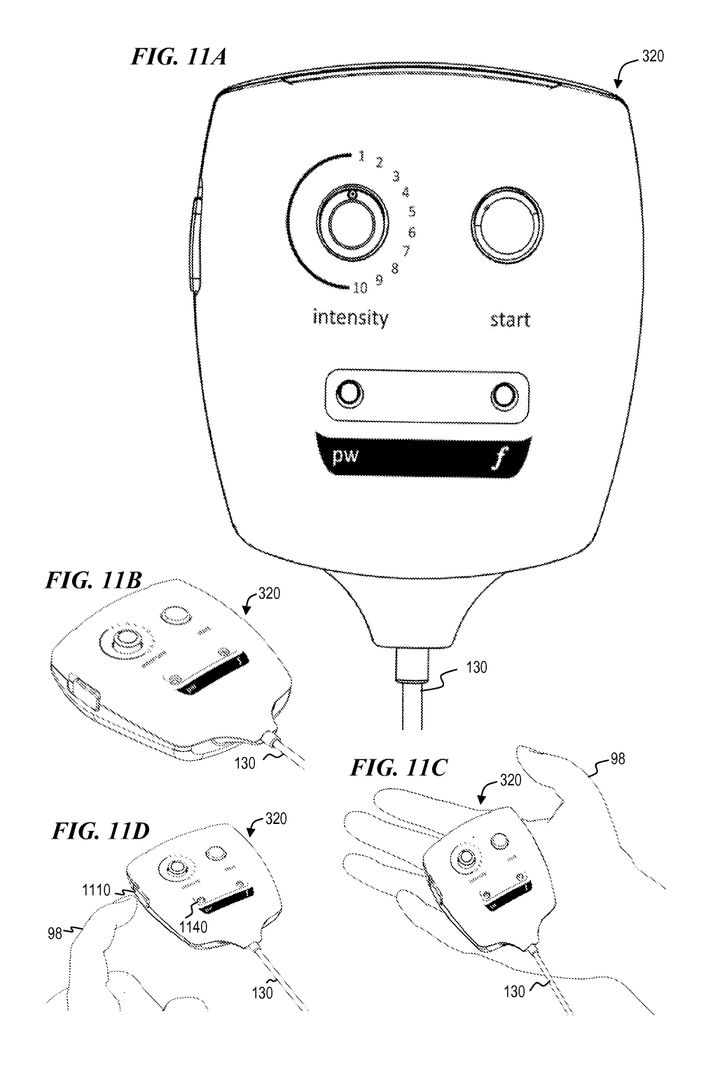

FIG. 11A is a front view of controller/power module 320, according to some embodiments of the invention. FIG. 11B is a front-perspective view of controller/power module 320, according to some embodiments of the invention.

FIG. 11C is a front-perspective view of controller/power module 320 in the hand of operator 98, according to some embodiments. FIG. 11D is a front-perspective view of controller/power module 320 that illustrates operator 98 pressing the power-on/off button 1110 of controller/power module 320, according to some embodiments. In some embodiments, pressing the power-on/off button 1110 once turns on the power, and pressing button 1110 again turns off the power. In some embodiments, LED 1140 turns on to indicate when the power is turned on.

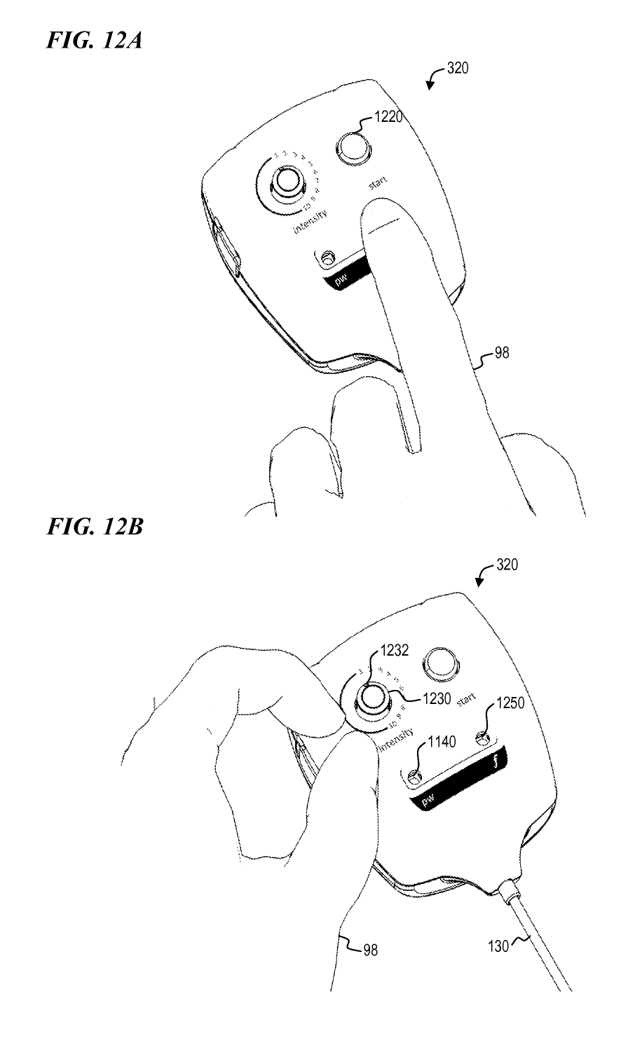

FIG. 12A is a front-perspective view of controller/power module 320 that illustrates operator 98 about ready to press the start button 1220, according to some embodiments of the invention. When the power is turned on and the start button 1220 is pressed, electrical stimulation transmitted to the head piece 110. In some embodiments, when the start button 1220 is pressed a second time, electrical stimulation ceases. LED 1250 (shown in FIG. 12B) is used to indicate when electrical stimulation transmitted to the head piece 110. In some embodiments, LED 1250 flashes to indicate when electrical stimulation transmitted to the head piece 110. The operator 98 may be the patient 99, but could be another person controlling the TENS unit for the patient 99. FIG. 12B is a front-perspective view of controller/power module 320 that illustrates operator 98 about ready to adjust the intensity button 1230, according to some embodiments of the invention. The intensity button is adjustable from low to high (e.g., 1 to 10). The adjustable dial has an indicator 1232 to show the present level of intensity.

In some embodiments of each of the embodiments described herein, a wirelessly coupled remote control (e.g., Bluetooth® or ZigBee® or the like) is used in place of or in addition to wired controls shown and described.

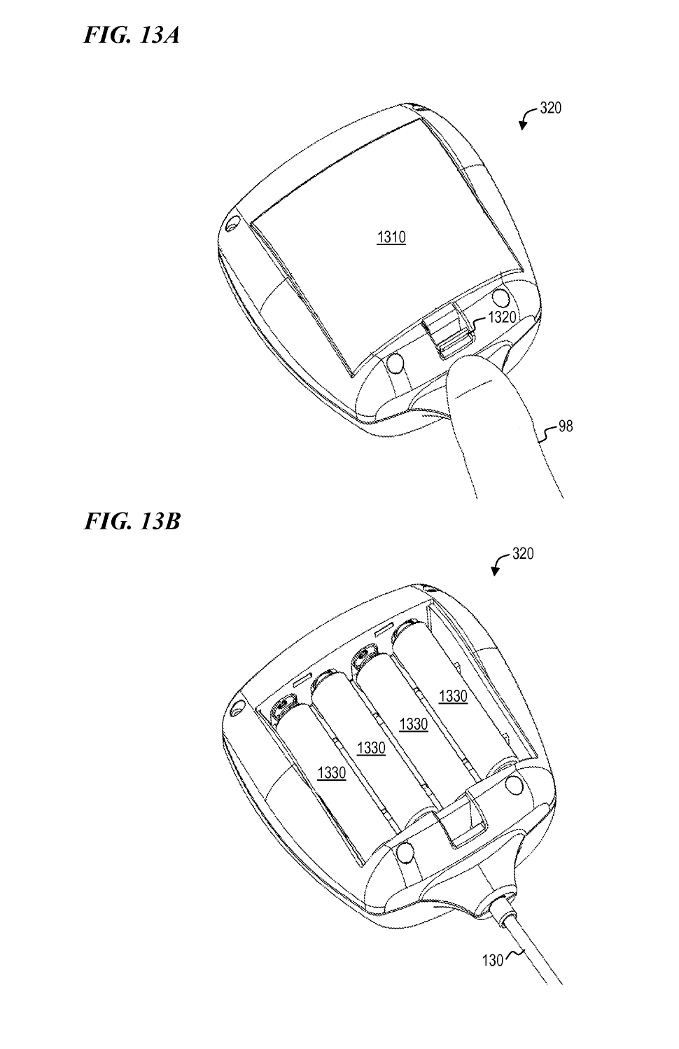

FIG. 13A is a back-perspective view of controller/power module 320 that illustrates operator 98 about ready to release the latch 1320 on battery-compartment cover 1310, according to some embodiments of the invention. FIG. 13B is a back-perspective view of controller/power module 320 that illustrates battery-compartment cover 1310 removed to provide access to batteries 1330, according to some embodiments of the invention.

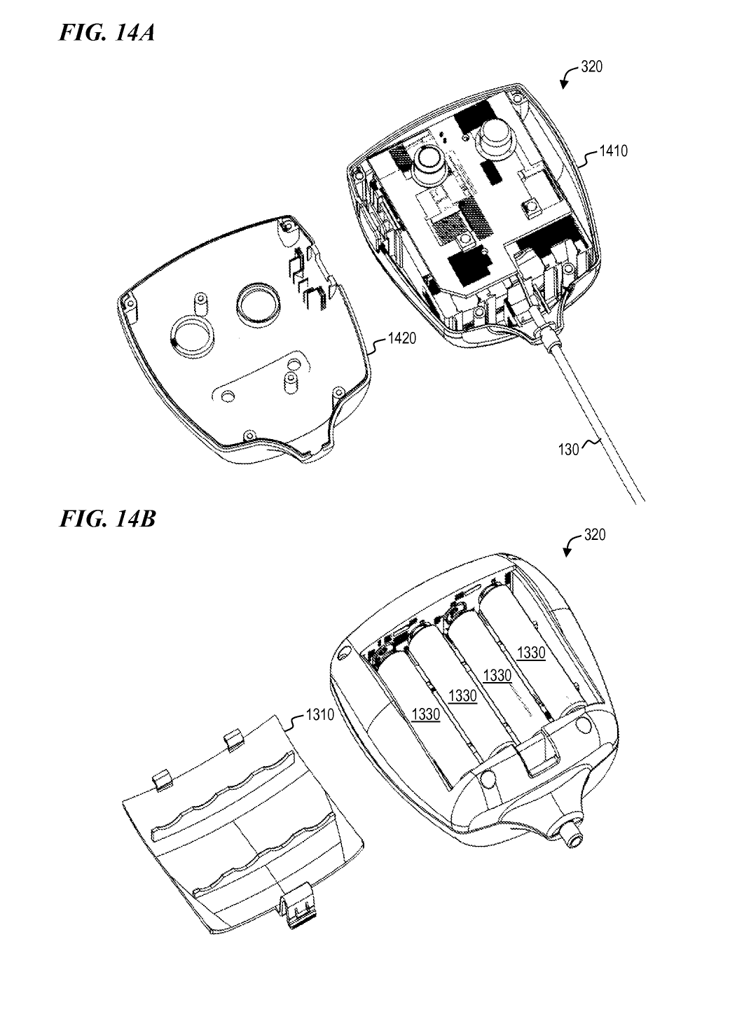

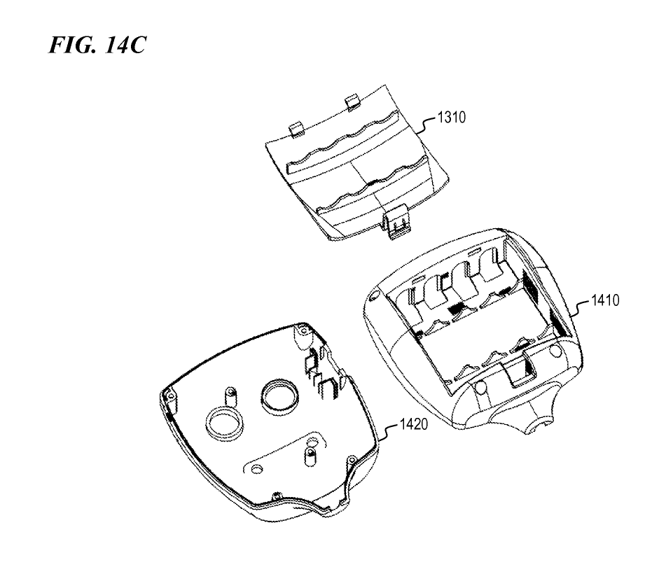

FIG. 14A is a front-perspective view of controller/power module 320 that illustrates module body 1410 and internal electronics with front cover 1420 removed, according to some embodiments of the invention. FIG. 14B is a back-perspective view of controller/power module 320 that illustrates battery-compartment cover 1310 removed to provide access to batteries 1330, according to some embodiments. FIG. 14C is a back-perspective view of controller/power module body 1410, front cover 1420, and battery-compartment cover 1310, according to some embodiments. In some embodiments, controller/power module body 1410, front cover 1420, and battery-compartment cover 1310 are molded from plastic or a polymer.

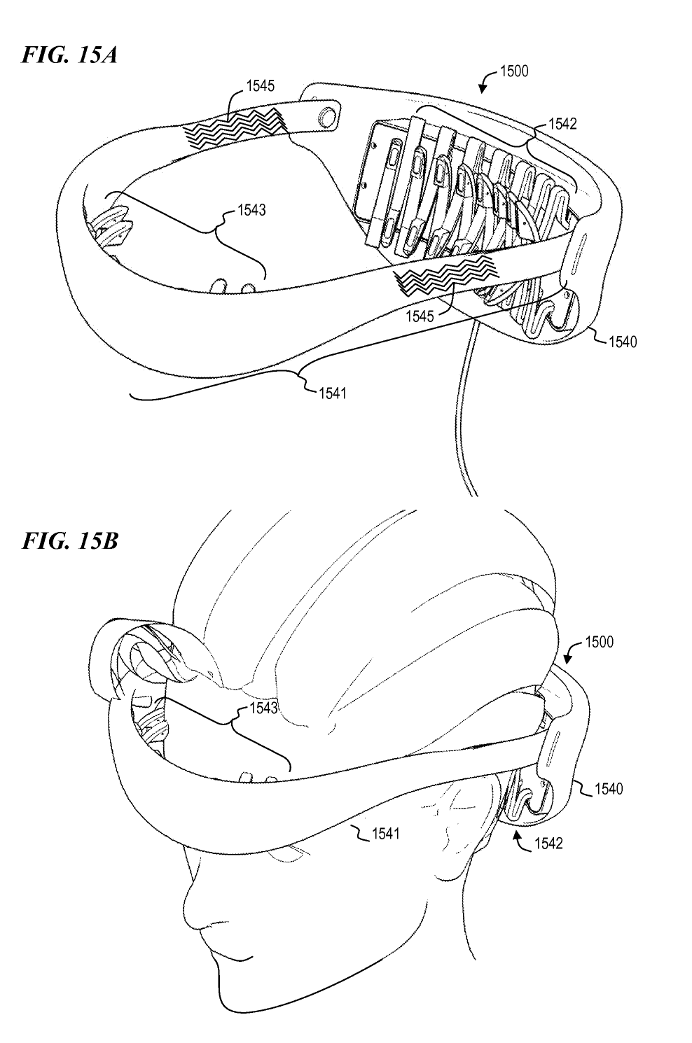

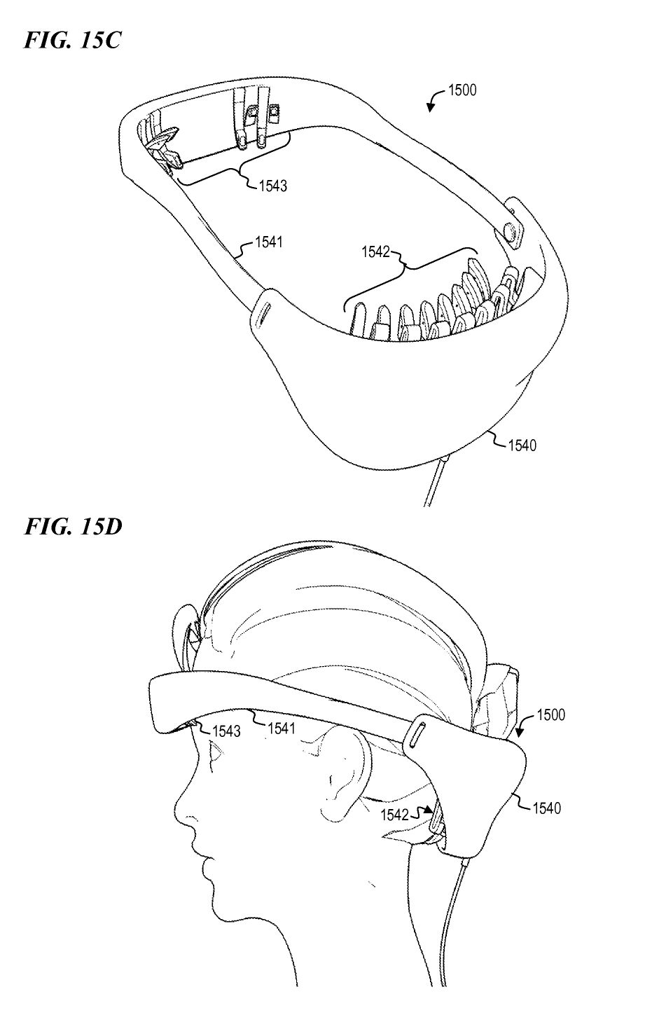

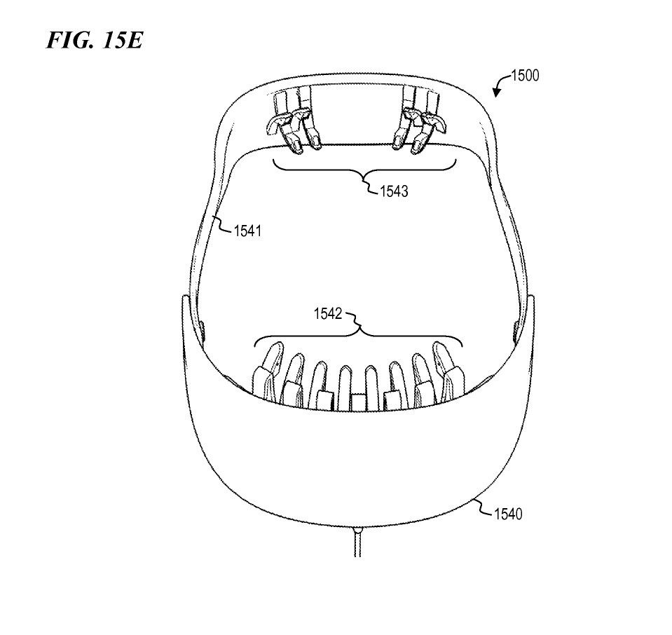

FIG. 15A is a front-left-perspective view of a combined occipital-and-supraorbital transcutaneous nerve stimulator unit 1500, according to some embodiments of the invention. In some embodiments, the combined occipital-and-supraorbital transcutaneous nerve stimulator unit 1500 includes occipital head piece 1540, and supraorbital head piece 1541. In some embodiments, the occipital head piece 1540 includes spring electrodes 1542, similar to spring electrodes 142 described earlier. In some embodiments, the supraorbital head piece 1541 includes spring electrodes 1543, similar to spring electrodes 142 described earlier, wherein sets of spring electrodes 1543 are spaced to contact the patient on the skin above the eyes over the supraorbital nerves. In some embodiments, spring electrodes 1542 and spring electrodes 1543 are driven by systems 1001 as described for FIG. 10A or systems 1002 as described for FIG. 10B. In some embodiments, the supraorbital head piece 1541 includes adjustable or elastic side pieces 1545 to provide sufficient tension to hold nerve stimulator unit 1500 in place on the patient's head and to provide electrical contact with spring electrodes 1542 and spring electrodes 1543, without causing discomfort to the patient.

FIG. 15B is a front-left-perspective view of combined occipital-and-supraorbital transcutaneous nerve stimulator unit 1500 in place on both the front and back of the head of patient 99, according to some embodiments of the invention. FIG. 15B shows spring electrodes 1543 in contact with the patient on the skin above the eyes over the supraorbital nerves.

FIG. 15C is a back-left-perspective view of combined occipital-and-supraorbital transcutaneous nerve stimulator unit 1500, according to some embodiments of the invention.

FIG. 15D is a side-perspective view of combined occipital-and-supraorbital transcutaneous nerve stimulator unit 1500 in place on both the front and back of the head of patient 99, according to some embodiments of the invention.

FIG. 15E is a back-perspective view of combined occipital-and-supraorbital transcutaneous nerve stimulator unit 1500, according to some embodiments of the invention.

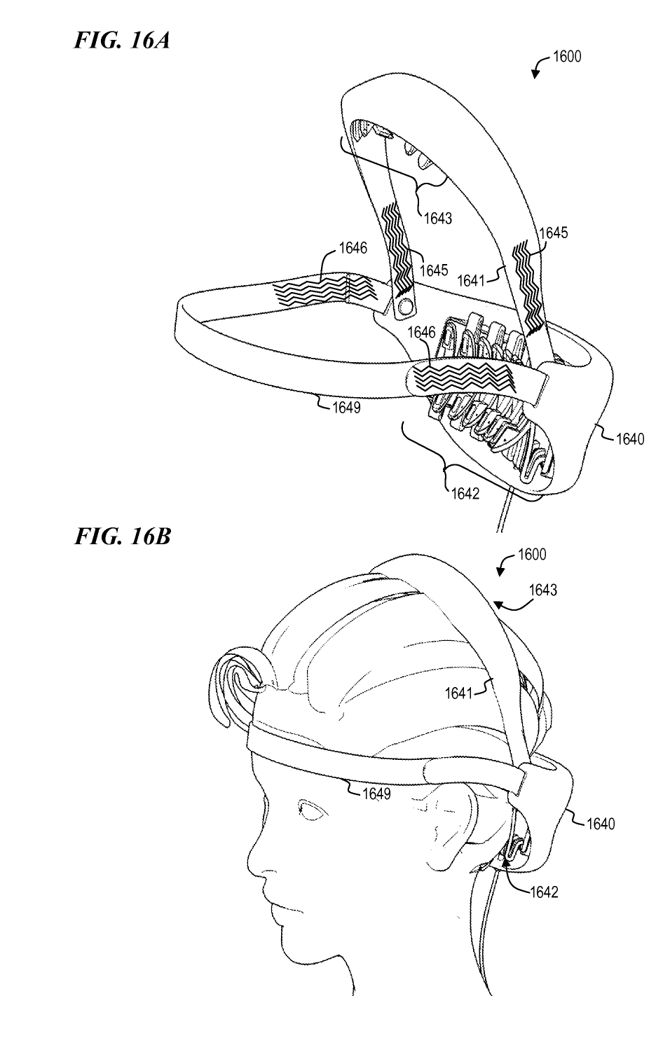

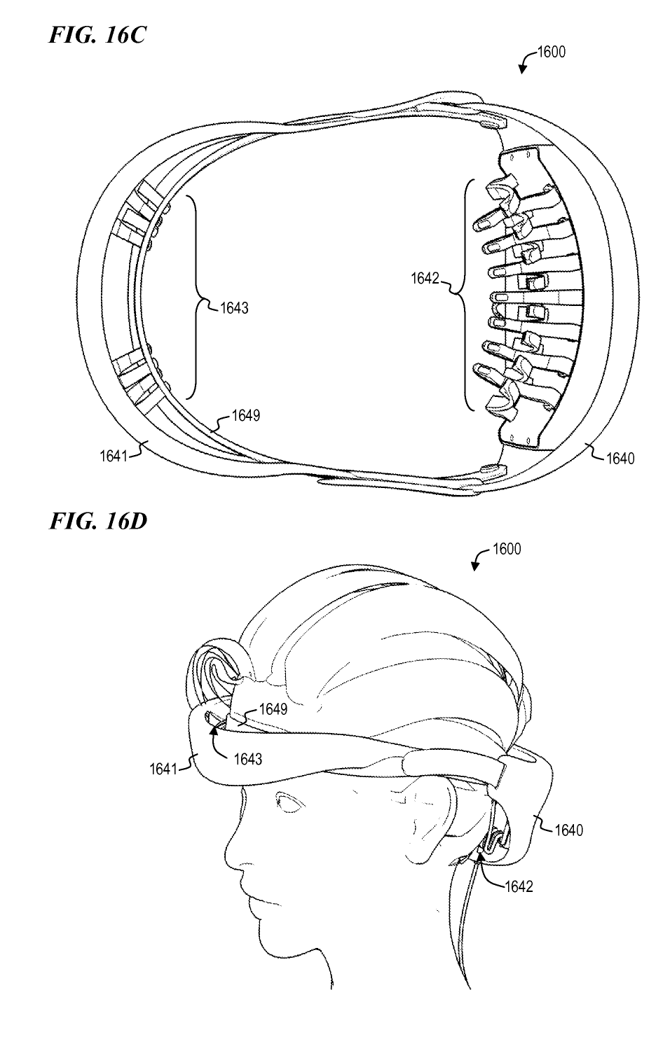

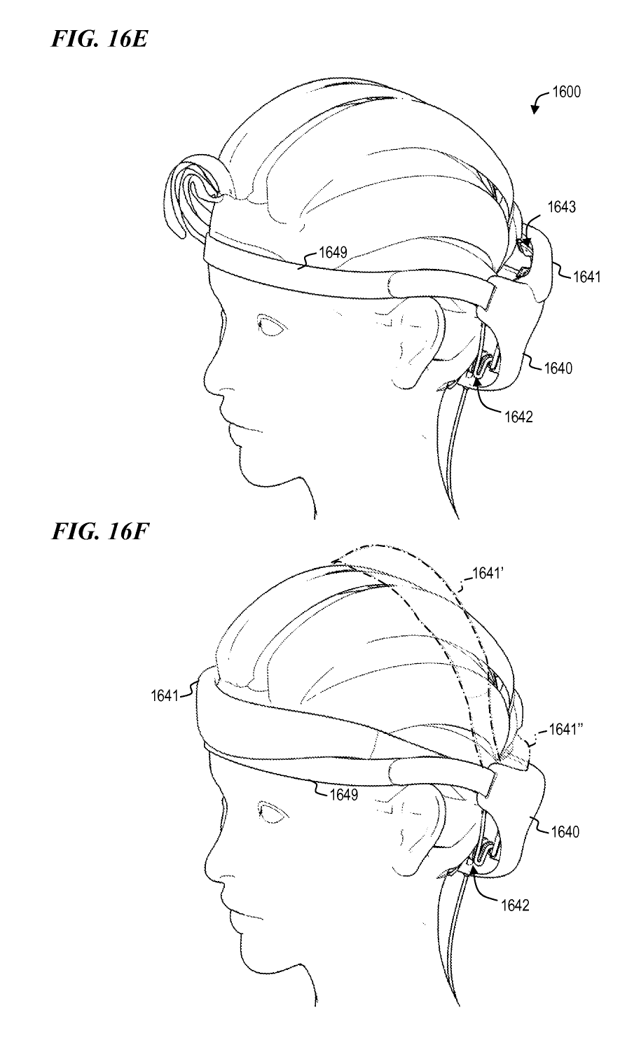

FIG. 16A is a front-left-perspective view of a combined occipital-and-supraorbital transcutaneous nerve stimulator unit 1600 having a separate placement-holder 1649, according to some embodiments. In some embodiments, the combined occipital-and-supraorbital transcutaneous nerve stimulator unit 1600 is similar to nerve stimulator unit 1500, and includes occipital head piece 1640, and supraorbital head piece 1641. In some embodiments, the occipital head piece 1640 includes spring electrodes 1642 and supraorbital head piece 1641 includes spring electrodes 1643. In some embodiments, spring electrodes 1642 and spring electrodes 1643 are driven by systems such as system 1001 as described for FIG. 10A or system 1002 as described for FIG. 10B. In some embodiments, the nerve stimulator unit 1600 includes placement-holder 1649 with adjustable and/or elastic pieces 1646 to provide sufficient tension to hold or maintain nerve stimulator unit 1600 in place on the patient's head and provide good electrical contact with spring electrodes 1642, without causing discomfort to the patient. In some embodiments, the supraorbital head piece 1641 includes and adjustable or elastic strap(s) 1645 with sufficient tension to provide good electrical contact between the patient and spring electrodes 1543, while not causing discomfort to the patient.

FIG. 16B is a front-left-perspective view of combined occipital-and-supraorbital transcutaneous nerve stimulator unit 1600 having a separate placement-holder 1649 holding the back portion 1640 in place on the back of the head of patient 99, according to some embodiments. In FIG. 16B, the transcutaneous nerve stimulator unit 1600 is shown with the placement-holder 1649 in place on the patient. Also shown in FIG. 16B, supraorbital head piece 1641 ready to be lowered into place over the supraorbital region of the forehead.

FIG. 16C is a top-perspective view of combined occipital-and-supraorbital transcutaneous nerve stimulator unit 1600 having a separate placement-holder 1649, according to some embodiments. FIG. 16D is a side-perspective view of combined occipital-and-supraorbital transcutaneous nerve stimulator unit 1600 having a separate placement-holder 1649 with front unit 1641 and back unit 1640 both in place on both the front and back of the head of patient 99, according to some embodiments. FIG. 16E is a front-left-perspective view of combined occipital-and-supraorbital transcutaneous nerve stimulator unit 1600 having a separate placement-holder 1649 holding the back portion 1640 in place on the back of the head of patient 99 with front unit 1641 resting on back unit 1640, according to some embodiments. FIG. 16F is a front-left-perspective view of combined occipital-and-supraorbital transcutaneous nerve stimulator unit 1600 having a separate placement-holder 1649 holding the back portion 1640 in place on the back of the head of patient 99 with front unit 1641 shown in place in the front of the patient's head and in phantom lines in intermediate positions from resting on top of back unit 1640, according to some embodiments of the invention.

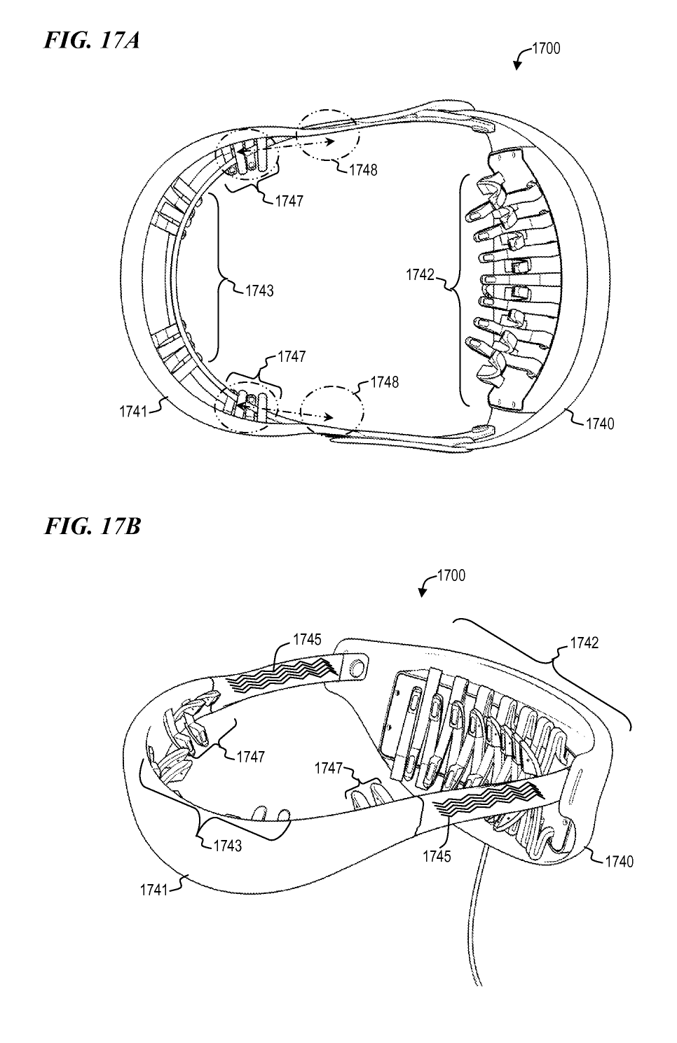

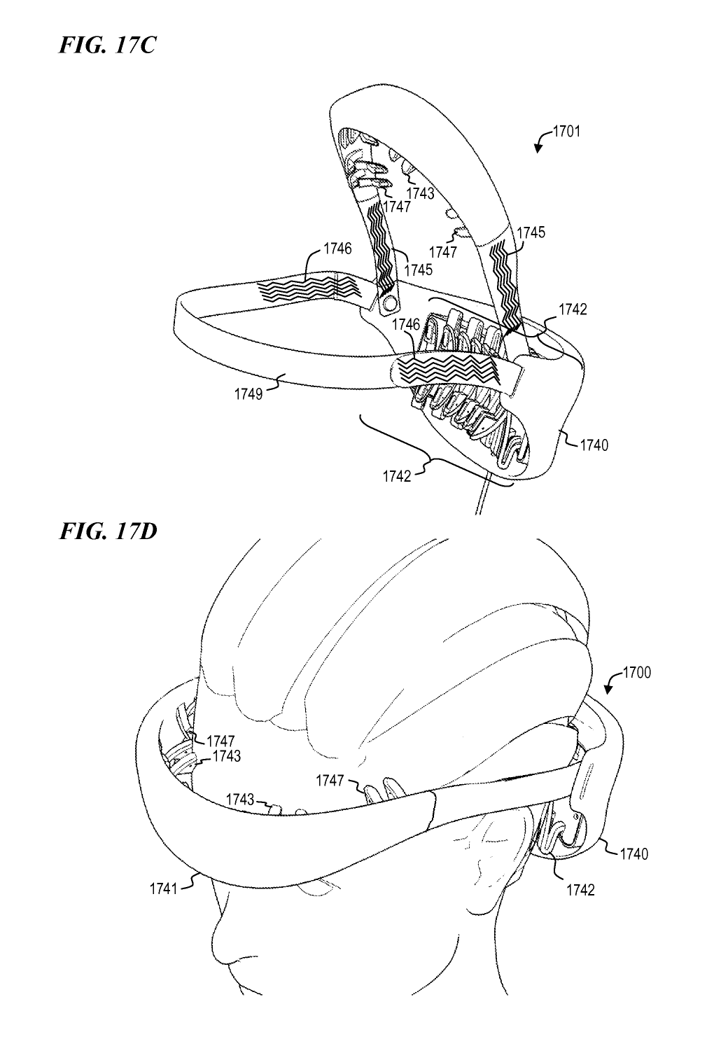

FIG. 17A is a top-perspective view of combined occipital-and-supraorbital-and-temporal transcutaneous nerve stimulator unit 1700 having additional side electrodes 1747 on either side of the front electrodes 1743 on front unit 1741, according to some embodiments of the invention. In some embodiments, the additional side electrodes 1747 are placed instead at about location 1748. In some embodiments, the position of additional side electrodes 1747 is adjustable from the location shown for electrodes 1747 to location 1748 or locations in between, or to locations further toward the occiput of the head. In some embodiments, the combined occipital-and-supraorbital-and-temporal transcutaneous nerve stimulator unit 1700 is similar to nerve stimulator unit 1600, and includes occipital head piece 1740, and supraorbital head piece 1741. In some embodiments, the occipital head piece 1740 includes spring electrodes 1742, supraorbital head piece 1741 includes spring electrodes 1743 and temporal spring electrodes 1747. In some embodiments, spring electrodes 1742, 1743, and 1747 are driven by systems such as system 1001 as described for FIG. 10A or system 1002 as described for FIG. 10B. In some embodiments, the supraorbital head piece 1741 includes adjustable or elastic side pieces 1745 to provide sufficient tension to hold nerve stimulator unit 1700 in place on the patient's head and to provide electrical contact with spring electrodes 1742, spring electrodes 1743, and spring electrodes 1747, without causing discomfort to the patient.

FIG. 17B is a front-left-perspective view of combined occipital-and-supraorbital-and-temporal transcutaneous nerve stimulator unit 1700, according to some embodiments of the invention. FIG. 17C is a front-left-perspective view of combined occipital-and-supraorbital-and-temporal transcutaneous nerve stimulator unit 1701 having a separate placement-holder 1749 and having additional side electrodes 1747 on either side of the front electrodes 1743 on front unit 1741, according to some embodiments of the invention. FIG. 17D is a front-left-perspective view of combined occipital-and-supraorbital-and-temporal transcutaneous nerve stimulator unit 1700 having a separate placement-holder 1749 holding the back portion 1740 in place on the back of the head of patient 99 with front unit 1741 shown in place in the front of the patient's head, according to some embodiments.

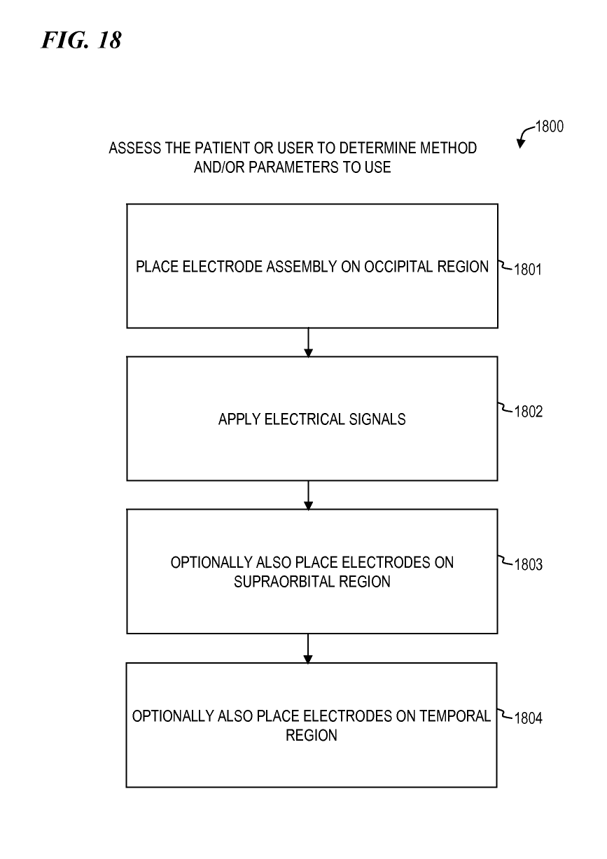

FIG. 18 is a flow chart of a method 1800, according to some embodiments of the invention. In some embodiments, method 1800 is used to treat headache. In some embodiments, method 1800 is used to treat a neuropsychiatric and/or neurological condition. In some embodiments, method 1800 includes the operations of placing 1801 one of the electrode assemblies (e.g., assembly as described herein) on patient's head with a plurality of electrodes in contact with the occipital portion of head (also called the occipital region), and applying 1802 electrical signals to the plurality of electrodes in contact with the occipital portion of head, Some embodiments further include optionally also placing and applying 1803 electrical signals to a plurality of electrodes in contact with one or more supraorbital portions of the patient's head (also called the supraorbital region), and such that the electrical signals are also applied to the plurality of electrodes in contact with the one or more supraorbital portions of the head. Some embodiments further include optionally also placing and applying 1804 electrical signals to a plurality of electrodes in contact with one or more temporal portions of the patient's head (also called the temporal region), and such that the electrical signals are also applied to the plurality of electrodes in contact with the one or more temporal portions of the head.

In some embodiments, individual pairs of electrodes (each pair including an upward-positioned electrode and a downward-positioned electrode) are separately activatable such that electrical stimulation signals to different pairs are applied in electrical isolation one-to-another (either by using time-wise separation or by providing electrically isolated pairs of signals such that electricity flows preferentially or only in an intra-pair path rather than flowing between a positive electrode in one pair of electrodes to a negative electrode in a different unintended or remote pair of electrodes. In some embodiments, a bi-phasic signal is used (a signal having one or more positive-going pulses and an equivalent one or more negative-going pulses, wherein the charge delivered in the positive pulses is substantially equal to the charge delivered in the negative pulses, in order to prevent accumulation of charge.

In some embodiments, the method 1800 is used to treat headache and/or a headache-related disorder. In some embodiments, the method 1800 is alternatively or also used to treat depression, epilepsy, anxiety, post-traumatic stress disorder (PTSD), and behavioral disorders (collectively, neuropsychiatric and/or neurological conditions) via transcutaneous stimulation of the occipital area of the head while also transcutaneously stimulating one or more portions of the trigeminal nerve. In some embodiments, transcutaneous stimulation of one or more branches of the trigeminal nerve located on the head, including one or more of supraorbital, supratrochlear, infraorbital, auriculotemporal, zygomaticotemporal, zygomaticoorbital, zygomaticofacial, infraorbital, nasal and mentalis nerves (also referred to collectively as the trigeminal nerve).

In some embodiments, the method 1800 is used to treat epilepsy and/or an epilepsy-related disorder and/or seizure-related disorder. In some embodiments, the method 1800 is used to treat depression and/or a depression-related disorder. In some embodiments, the method 1800 is used to treat anxiety and/or an anxiety-related disorder. In some embodiments, the method 1800 is used to treat post-traumatic stress disorder. In some embodiments, the method 1800 is also used to adjust the user's alertness and/or wakefullness. In some embodiments, the method 1800 is also used to address or treat attention deficit hyperactivity disorder (ADHD).

In some embodiments, the method 1800 is used to treat an alertness condition or disorder. In some embodiments, an additional action of assessing one or more conditions of a patient or user of the method 1800 precedes the placing 1801, in order to determine the appropriateness of the method and/or to adjust various parameters of the method such as the location(s) at which to apply the signal(s), and/or the pulse parameters (such as voltage, current, pulse shape, pulse repetition rate, modulation frequency, the amounts/durations of the positive and negative portions of a biphasic pulse, and the like. In some embodiments, the method 1800 is also used to treat an alertness condition or disorder (e.g., sleepiness, narcolepsy, etc.) via transcutaneous stimulation of the occipital area of the head while also transcutaneously stimulating one or more portions of the trigeminal nerve. In some embodiments, transcutaneous stimulation of one or more branches of the trigeminal nerve located on the head, including one or more of supraorbital, supratrochlear, infraorbital, auriculotemporal, zygomaticotemporal, zygomaticoorbital, zygomaticofacial, infraorbital, nasal and mentalis nerves (also referred to collectively as the trigeminal nerve).

In some embodiments, the method 1800 is used to treat ADHD. In some embodiments, an additional action of assessing one or more conditions of a patient or user of the method 1800 precedes the placing 1801, in order to determine the appropriateness of the method to treat ADHD and/or to adjust various parameters of the method such as the location(s) at which to apply the signal(s), and/or the pulse parameters (such as voltage, current, pulse shape, pulse repetition rate, modulation frequency, the amounts/durations of the positive and negative portions of a biphasic pulse, and the like).

In some embodiments, the method 1800 is also used to treat ADHD via transcutaneous stimulation of the occipital area of the head while also transcutaneously stimulating one or more portions of the trigeminal nerve. In some embodiments, transcutaneous stimulation of one or more branches of the trigeminal nerve located on the head, including one or more of supraorbital, supratrochlear, infraorbital, auriculotemporal, zygomaticotemporal, zygomaticoorbital, zygomaticofacial, infraorbital, nasal and mentalis nerves (also referred to collectively as the trigeminal nerve).

In some embodiments of method 1800 of FIG. 18, the present invention supplements or replaces the electrical signals of the plurality of electrodes with a plurality of ultrasonic signals applied at like positions of the head of the user. In some embodiments, the methods using ultrasound apply the ultrasound from small ultrasound transducers of the approximate size and shape of the electrode tips of the apparatus shown in FIGS. 1A-17D. In some embodiments, these ultrasound transducers are powered by electrical signals applied through conductive paths in a manner substantially the same as described for the electrode signals, except that for ultrasound transducers a pair of wires delivers the electrical signals needed to drive the transducers. In some embodiments, the ultrasound is pulsed in a manner similar to the pulsing of the electrical signals. In other embodiments, a continuous-wave (CW) or quasi-CW ultrasound signal is used. In some embodiments, both electrical and ultrasound signals are applied to the head of the user. In some embodiments, the same tooth is used both as an electrode to apply the electrical signal and as an ultrasound transducer to apply the ultrasound signal. In some embodiments, the gel applicators of the present invention facilitate the transfer of gel to assist ultrasound signals as well as or in place of the gel electrolyte that facilitates the electrical conduction.

In some embodiments, the present invention provides an apparatus for occipital and optionally for trigeminal nerve stimulation for treatment of neuropsychiatric, neurological, and alertness conditions or disorders, and/or attention deficit hyperactivity disorder (ADHD). In some embodiments, the apparatus includes a pulse generator; and a transcutaneous electrode unit operably connected to the pulse generator. The transcutaneous electrode unit includes a plurality of electrode pairs including a first electrode pair and a second electrode pair, each electrode pair having at least two electrical contacts, wherein the first electrode pair is configured to be placed at a first region of the patient's head overlying one or more occipital nerves, and wherein the second electrode pair is configured to be placed at a second region of the patient's head overlying one or more branch of the trigeminal nerve. In some embodiments, the one or more branches of the trigeminal nerve are selected from the group consisting of the auriculotemporal nerve(s) and the supraorbital nerve(s). In some embodiments, the one or more occipital nerve are selected from the group consisting of the greater occipital nerve(s), the lesser occipital nerve(s), the third occipital nerve(s), greater auricular nerve(s), transverse cervical nerve(s), the supraclavicular nerve(s), and/or branches of any of these nerves.

In some embodiments, the one or more branches of the trigeminal nerve are selected from the group consisting of the auriculotemporal nerve, the infraorbital nerve, the infratrochlear nerve, the mentalis nerve, the nasal nerve, the ophthalmic nerve, the supratrochlear nerve, the zygomaticofacial nerve, the zygomaticoorbital nerve, the zygomaticotemporal nerve.

In some embodiments, the present invention provides a method that includes providing a transcutaneous electrical nerve stimulator (TENS) headache-treatment system having a first plurality of projecting spring electrodes; providing a sealed gel dispenser having a plurality of pockets each containing an electrically conductive gel, the gel-containing pockets being covered by a membrane seal; unsealing the sealed gel dispenser; and applying the gel from the dispenser substantially simultaneously to a distal end of each of the first plurality of projecting spring electrodes. In some embodiments, the unsealing of the sealed gel dispenser includes puncturing the seal on the gel dispenser with the first plurality of projecting spring electrodes such that the gel is applied substantially simultaneously to the first plurality of projecting spring electrodes. In some embodiments, the sealed gel dispenser includes a separable peel-off protective layer, and the method further includes manually removing at least a portion of the peel-off layer to expose the seal on the gel dispenser before puncturing the seal on the gel dispenser with the first plurality of projecting spring electrodes. In some embodiments, the unsealing of the sealed gel dispenser includes manually removing a separable peel-off protective layer. Some embodiments further include absorbing the conductive gel into a sponge-like material on the tips of the projecting spring electrodes.

In some embodiments, the present invention provides a headache-treatment system that includes an electrode base shaped to conform to a back of a human user's head; a transcutaneous electrical nerve stimulator (TENS) having a first plurality of projecting spring electrodes each physically connected to the electrode base; and a gel dispenser or pack that holds an electrically conductive gel in each of a plurality of sealed pockets of the dispenser; wherein the plurality of projecting spring electrodes and the gel pack are configured such that pressing the plurality of projecting spring electrodes and the gel pack against one another both unseals the gel pack and applies the gel substantially simultaneously to the first plurality of projecting spring electrodes. In some embodiments of the headache-treatment system of the present invention, the gel dispenser includes a membrane seal covering the electrically conductive gel in the plurality of sealed pockets, and wherein the projecting spring electrodes are configured to substantially simultaneously puncture the membrane seal on a plurality of the pockets of the gel dispenser such that the gel is applied substantially simultaneously to the first plurality of projecting spring electrodes. In some such embodiments, the gel dispenser includes a separable peel-off protective layer covering the membrane seal and configured to be manually removed before puncturing the seal of the gel dispenser with the first plurality of projecting spring electrodes.

In some embodiments, the present invention provides a headache-treatment system that includes an electrode base shaped to conform to a back of a human user's head; a transcutaneous electrical nerve stimulator (TENS) having a first plurality of projecting spring electrodes each physically connected to the electrode base; and a gel dispenser or pack that holds an electrically conductive gel in each of a plurality of sealed pockets of the dispenser; wherein the gel pack has a manually removable membrane seal, and once the seal is removed the gel is exposed, and the plurality of projecting spring electrodes and the gel pack are configured such that pressing them against one another applies the gel substantially simultaneously to the first plurality of projecting spring electrodes. Some embodiments of the headache-treatment system further includes a sponge-like material affixed on the tips of the projecting spring electrodes, wherein the sponge-like material is configured to absorb and/or wick the conductive gel into the sponge-like material. In some embodiments of the system, the sealed gel dispenser includes a flexible plastic material having a plurality of blisters formed therein, each blister forming one of the plurality of pockets and containing the electrically conductive gel; and at least one protective seal layer affixed to the flexible plastic material around each of the plurality of pockets, wherein the seal layer extends over and covers the gel held in the plurality of blisters.

In some embodiments of the headache-treatment system, the electrode base includes a first substantially enclosed electrically conductive busbar, and each of the first plurality of projecting spring electrodes includes an inner-spring metal conductor that is covered for a middle length of the spring electrode by a polymer sheath, the inner-spring metal conductor of each of the first plurality of projecting spring electrodes is coupled to the first conductive busbar at a first end of the spring electrode within the electrode base, and each of the first plurality of projecting spring electrodes includes a sponge or sponge-like material at a second end, and wherein the sponge or sponge-like material is connected to the inner-spring metal conductor. In some embodiments of the system, the inner-spring metal conductor includes stainless steel.

In some embodiments, the present invention provides a headache-treatment system that includes an electrode base shaped to conform to a back of a human user's head; a transcutaneous electrical nerve stimulator (TENS) having a first plurality of projecting spring electrodes each physically connected to the electrode base; means for holding an electrically conductive gel in a plurality of sealed pockets; and means for unsealing the means for holding the gel and applying the gel substantially simultaneously to a distal end of each of the first plurality of projecting spring electrodes. In some embodiments, the means for unsealing the means for holding the gel and for applying the gel includes means for puncturing a sealed membrane on the means for holding the gel with the first plurality of projecting spring electrodes such that the gel is applied substantially simultaneously to the first plurality of projecting spring electrodes.

In some embodiments, the means for holding the gel includes a separate peel-off protective layer manually removed before puncturing the seal on the gel dispenser with the first plurality of projecting spring electrodes. In some embodiments, the means for unsealing the means for holding the gel includes a manually removable separable peel-off protective layer. Some embodiments further include a sponge-like material affixed on the tips of the projecting spring electrodes to absorb the conductive gel. In some embodiments, the sealed gel dispenser includes a flexible plastic material having a plurality of blisters formed therein, each blister forming one of the plurality of pockets and containing the electrically conductive gel; and at least one protective seal layer over each of the plurality of pockets. In some embodiments, the electrode base includes a first substantially enclosed electrically conductive busbar; each of the first plurality of projecting spring electrodes includes an inner spring-metal conductor that is covered for a middle length of the spring electrode by a polymer sheath; the inner spring-metal conductor of each of the first plurality of projecting spring electrodes is coupled to the first conductive busbar at a first end of the spring electrode within the electrode base; each of the first plurality of spring electrodes includes a sponge or sponge-like material at a second end; and the sponge or sponge-like material is electrically connected to the inner-spring metal conductor. In some embodiments of the apparatus, the inner-spring metal conductor includes stainless steel.

In some embodiments, the present invention provides a dispenser apparatus (e.g., in some embodiments, as part of a kit) that includes a gel dispenser having a plurality pockets each containing an electrically conductive gel, wherein the gel dispenser is sealed with a readily penetrable and/or puncturable membrane. In some embodiments of the dispenser apparatus, the membrane includes a metal foil and/or a foil-like material. In some embodiments, the membrane is manually peelable from the pockets. In some embodiments, the present invention provides a dispenser apparatus for dispensing gel electrolyte. This apparatus includes a sealed gel dispenser having a plurality of pockets each containing an electrically conductive gel electrolyte; and at least one membrane affixed to a surface of the gel dispenser and across the plurality of pockets, such that the gel dispenser is sealed with the at least one membrane. In some embodiments of the dispenser apparatus, the at least one membrane includes a metal foil and/or a foil-like material (e.g., a metal-coated polymer film such as aluminum-coated Mylar® or the like). In some embodiments, the at least one membrane includes a manually peelable membrane that has a graspable tab that can be pulled by the user to remove the membrane and expose the gel.

In some embodiments of the dispenser apparatus, the at least one membrane includes a manually peelable protective membrane or cover (such as tag board or cardboard or other stiff paper product) that is affixed over a readily-puncturable membrane (such as metal foil). In some embodiments, the foil layer is used to seal the gel and prevent drying out or contamination of the gel, and the protective cardboard layer prevents inadvertent puncturing of the foil layer. In some embodiments, the foil is readily puncturable. That is, the foil is puncturable by application of a force of about 10 Newtons (about 2.25 pounds) or less. In other embodiments, the foil is more readily puncturable by application of a force of about 5 Newtons (about 1.125 pounds) or less. In other embodiments, the foil is readily even more puncturable by application of a force of about 1 Newton (about 0.225 pounds) or less. In some embodiments, the electrode cover 416 is configured to reduce the amount of force needed to puncture the foil by being shaped such that the electrode cover 416 applies pressure to fewer than all the gel pockets at any one time (e.g., electrode cover 416 can be “rolled” across the gel pack such that one to four pockets are simultaneously punctured) but the cover still allows all the pockets to be punctured in a sequence that takes less than five seconds, or even less than two seconds of a rolling motion. Thus, the foil-covered gel pack 550 is readily puncturable.

Some embodiments of the dispenser apparatus further include a headache-treatment system that includes an electrode base shaped to conform to a back of a human user's head; a transcutaneous electrical nerve stimulator (TENS) unit having a first plurality of projecting spring electrodes each physically connected to the electrode base, and a control unit that is communicatively coupled to control operation of the TENS unit. In some such embodiments, the TENS unit further includes a hands-free strap connected to the electrode base and that holds the spring electrodes against the back of the human user's head.

REFERENCES CITED: The following relate to the invention and all are incorporated herein by reference: U.S. Pat. No. 4,856,526, U.S. Pat. No. 4,627,438, and U.S. Patent Application Publication No. 2006/0173510. Other references are as follows:
BARTSCH, T. & P. J. GOADSBY; “Stimulation of the greater occipital nerve induces increased central excitability of dural afferent input”; Brain (2002) 125:1496-1509.
BUCHGREITZ, L., A. C. LYNGBERG, L. BENDTSEN & R. JENSEN; “Increased prevalence of tension-type headache over a 12-year period is related to increased pain sensitivity. A population study”; Cephalalgia (2007) 27:145-152.
CAROLEI, A., I. CIANCARELLI, D. CERONE & S. SACCO; “Comorbidities of migraine: a user-friendly overview”; Journal of Headache and Pain (2003) 4:S23-S25.
DARTIGUES, J. F., P. MICHEL & P. HENRY; “Evaluating the economic costs of migraine: interest of a comparative approach”; Journal of Headache and Pain (2003) 4:S63-S66.
IANNAZZO, S., M. S. CATTARUZZA, S. DE FILIPPIS, F. ROSSI, E. PERATA, S. DI ROLLO, G. COLOPRISCO & P. MARTELLETTI; “Analgesic therapy for headache: consumption, appropriateness and costs”; Journal of Headache and Pain (2003) 4:S84-S87.
JENSEN, R.; “Mechanisms of tension-type headache”; Cephalalgia (2001) 21:786-789.
JENSEN, R.; “Peripheral and central mechanisms in tension-type headache: an update”; Cephalalgia (2003) 23 (Suppl. 1) 49-52.
LIPTON, R. B., M. E. BIGAL, A. I. SCHER & W. F. STEWART; “The global burden of migraine”; Journal of Headache and Pain (2003) 4:S3-S11.
MARTELLETTI, P. & M. LEONARDI; “The global impact of migraine”; Journal of Headache and Pain (2003) 4:S1-S2.
MØRK, H., M. ASHINA, L. BENDTSEN, J. OLESEN & R. JENSEN; “Possible mechanisms of pain perception in patients with episodic tension-type headache. A new experimental model of myofascial pain”; Cephalalgia (2004) 24:466-475.
SOLOMON, S., P. KARFUNKEL & K. M. GUGLIELMO; “Migraine—Cluster Headache Syndrome”; Headache (1985) 25:236-239.
STOVNER, L., K. HAGEN, R. JENSEN, Z. KATSARAVA, R. LIPTON, A. SCHER, T. STEINER & J-A ZWART; “The global burden of headache: a documentation of headache prevalence and disability worldwide”; Cephalalgia (2007) 27:193-210.
TERZI, T., B. K ARAKURUM, S. ÜCLER, L. E. INAN & C. TULUNAY; “Greater occipital nerve blockade in migraine, tension-type headache and cervicogenic headache”; Journal of Headache and Pain (2002) 3:137-141.
VINDING, G. R., P. ZEEBERG, A. LYNGBERG, R. T. NIELSEN & R. JENSEN; “The burden of headache in a patient population from a specialized headache centre”; Cephalalgia (2007) 27: 263-270.

It is to be understood that the above description is intended to be illustrative, and not restrictive. Although numerous characteristics and advantages of various embodiments as described herein have been set forth in the foregoing description, together with details of the structure and function of various embodiments, many other embodiments and changes to details will be apparent to those of skill in the art upon reviewing the above description. The scope of the invention should be, therefore, determined with reference to the appended claims, along with the full scope of equivalents to which such claims are entitled. In the appended claims, the terms “including” and “in which” are used as the plain-English equivalents of the respective terms “comprising” and “wherein,” respectively. Moreover, the terms “first,” “second,” and “third,” etc., are used merely as labels, and are not intended to impose numerical requirements on their objects.

1. A method comprising:
providing a transcutaneous electrical nerve stimulator (TENS) headache-treatment system having a first plurality of projecting spring electrodes and operating the TENS with the first plurality of projecting spring electrodes in direct contact with a human user's head;
providing a sealed gel dispenser separate from the TENS headache-treatment system and having a first plurality of pockets each containing an electrically conductive gel, the gel-containing pockets being covered by a membrane seal;
unsealing the sealed gel dispenser;
applying the gel from the dispenser substantially simultaneously to a distal end of each of the first plurality of projecting spring electrodes; and
separating and detaching the first plurality of pockets from the first plurality of projecting spring electrodes after the gel is applied and before TENS is operated with the distal ends of the first plurality of projecting spring electrodes in direct contact with the human user's head.

2. The method of claim 1, wherein the unsealing the sealed gel dispenser includes puncturing the seal on the gel dispenser with the distal ends of the first plurality of projecting spring electrodes such that the gel is applied substantially simultaneously to the first plurality of projecting spring electrodes.

3. The method of claim 2, wherein the sealed gel dispenser includes a separable peel-off protective layer, and wherein the method further includes manually removing at least a portion of the peel-off layer before puncturing the seal on the gel dispenser with the first plurality of projecting spring electrodes.

4. The method of claim 1, wherein the unsealing the sealed gel dispenser includes manually removing a separate peel-off protective layer.

5. The method of claim 1, further comprising:
connecting a sponge-like material on the distal end of each of the first plurality of projecting spring electrodes; and
absorbing the conductive gel into the sponge-like material.

6. The method of claim 1, further comprising:
connecting an electrically conductive textile-like material on the distal end of each of the first plurality of projecting spring electrodes; and
absorbing the conductive gel into the sponge-like material.

7. An electrical-stimulation system comprising:
an electrode base shaped to conform to a back of a human user's head;
a transcutaneous electrical nerve stimulator (TENS) having a first plurality of projecting spring electrodes each connected to the electrode base on a proximal end, wherein the first plurality of projecting spring electrodes are configured to operate with a distal end in direct contact with the human user's head;
sealed means for holding a predetermined amount of electrically conductive gel in each of a first plurality of sealed pockets; and
means for unsealing each of the first plurality of sealed pockets of the means for holding the gel and for applying the gel substantially simultaneously to the distal end of each of the first plurality of projecting spring electrodes, and wherein the first plurality of pockets of the sealed means is separated from and detached from the first plurality of projecting spring electrodes after the applying of the gel and before TENS is operated with the first plurality of projecting spring electrodes in direct contact with the human user's head.

8. The system of claim 7, wherein the means for unsealing each of the first plurality of sealed pockets of the means for holding the gel and applying the gel includes means for puncturing a sealed membrane on each of the first plurality of sealed pockets of the means for holding the gel with the distal ends of the first plurality of projecting spring electrodes such that the gel is applied substantially simultaneously to the distal end of each of the first plurality of projecting spring electrodes.

9. The system of claim 8, wherein the means for holding the gel includes a separate peel-off protective layer configured to be manually removed before puncturing the seal on the gel dispenser with the first plurality of projecting spring electrodes.

10. The system of claim 7, wherein the means for unsealing the means for holding the gel and for applying the gel includes a separable peel-off protective-and-sealing layer configured to be manually removed such that once the protective-and-sealing layer is removed, the gel is unsealed such that when the means is placed against the first plurality of projecting spring electrodes, the gel is applied substantially simultaneously to the distal end of each of the first plurality of projecting spring electrodes.

11. The system of claim 7, further including a handheld remote control operatively coupled to the TENS to control an operation of the TENS.

12. The system of claim 11, wherein the handheld remote control is wirelessly coupled to the TENS to control an operation of the TENS.

13. The system of claim 7, wherein the sealed gel dispenser includes:
a flexible plastic material having a first plurality of blisters formed therein, each blister forming one of the first plurality of pockets and containing the electrically conductive gel; and
at least one protective seal layer over each of the first plurality of pockets.

14. The system of claim 7, wherein the electrode base includes a first substantially enclosed electrically conductive busbar, and wherein each of the first plurality of projecting spring electrodes includes an inner-spring metal conductor that is covered for a middle length of the spring electrode by a polymer sheath, wherein the inner-spring metal conductor of each of the first plurality of projecting spring electrodes is coupled to the first conductive busbar at a proximal end of the spring electrode within the electrode base, and wherein each of the first plurality of projecting spring electrodes includes a sponge or sponge-like material at the distal end, and wherein the sponge or sponge-like material is connected to the inner-spring metal conductor.

15. The system of claim 14, wherein the inner-spring metal conductor includes stainless steel.

16. The system of claim 7, further comprising an electrically conductive textile-like material on the distal end of each of the first plurality of projecting spring electrodes.

17. The system of claim 7, further including a hands-free means for holding the first plurality of projecting spring electrodes against the back of the human user's head.

18. The system of claim 7, further including a sponge-like material affixed on the distal end of each of the first plurality of projecting spring electrodes to absorb the conductive gel.

19. An apparatus for dispensing gel electrolyte to an operating surface of each one of a first plurality of spring electrodes, wherein the operating surfaces of the first plurality of spring electrodes operate in direct contact with a human user's head, the apparatus comprising:
a sealed gel dispenser having a first plurality of pockets each containing a predetermined amount of electrically conductive gel electrolyte; and
at least one membrane affixed to a surface of the gel dispenser and across the first plurality of pockets, such that the gel dispenser is sealed with the at least one membrane, and wherein each one of the first plurality of pockets dispenses gel to a respective one of the first plurality of spring electrodes and the dispenser is configured to be separated from and detached from the first plurality of spring electrodes after the gel is dispensed and before the first plurality of spring electrodes are placed in direct contact with the human user's head.

20. The apparatus of claim 19, wherein the at least one membrane includes a readily-puncturable membrane.

21. The apparatus of claim 20, wherein the readily-puncturable membrane includes a metal foil.

22. The apparatus of claim 19, wherein the at least one membrane includes a manually peelable membrane.

23. The apparatus of claim 19, wherein the at least one membrane includes a manually peelable membrane over a readily-puncturable membrane.

24. The apparatus of claim 19, further comprising a headache-treatment system that includes an electrode base shaped to conform to a back of a human user's head; a transcutaneous electrical nerve stimulator (TENS) unit having the first plurality of spring electrodes each physically connected to the electrode base, and a control unit that is communicatively coupled to control operation of the TENS unit.

25. The apparatus of claim 24, wherein the TENS unit further includes a hands-free strap connected to the electrode base and that holds the first plurality of spring electrodes against the back of the human user's head.

26. The system of claim 7, wherein each of the first plurality of projecting spring electrodes includes a leaf-type spring element.
